(19) United States
(12) Patent Application Publication
(10) Pub. No.: US 2026/0085334 A1
(43) Pub. Date: Mar. 26, 2026

(54) ENGINEERED KETOREDUCTASES FOR THE PREPARATION OF CHIRAL ALCOHOLS AND METHODS THEREOF

(71) Applicant: Enzymaster (Ningbo) Bio-Engineering Co., Ltd., Ningbo, Zhejiang (CN)

(72) Inventors: Haibin CHEN, Ningbo Zhejiang (CN); Yafang LIANG, Ningbo Zhejiang (CN); Yingxin ZHANG, Ningbo Zhejiang (CN); Zekun XIA, Ningbo Zhejiang (CN); Ameng ZHOU, Ningbo Zhejiang (CN); Marco BOCOLA, Ningbo Zhejiang (CN); Junpeng XU, Ningbo Zhejiang (CN); Xiao LUO, Ningbo Zhejiang (CN); Jingxian MAO, Ningbo Zhejiang (CN); Ruimei HONG, Ningbo Zhejiang (CN); Xinwei WU, Ningbo Zhejiang (CN); Lei SUN, Ningbo Zhejiang (CN); Yixin DAI, Ningbo Zhejiang (CN); Songxue WANG, Ningbo Zhejiang (CN); Qinli PENG, Ningbo Zhejiang (CN)

(73) Assignee: Enzymaster (Ningbo) Bio-Engineering Co., Ltd., Ningbo, Zhejiang (CN)

(21) Appl. No.: 18/879,783

(22) PCT Filed: May 17, 2023
(86) PCT No.: PCT/CN2023/094905

§ 371 Date: Dec. 29, 2024

(30) Foreign Application Priority Data
Jun. 29, 2022 (CN) 202210753932.8

(51) Int. Cl. C12P 7/62; C12N 1/20; C12N 9/04; C12N 11/00; C12N 15/52; C12N 15/70; C12P 7/02; C12P 13/02; C12R 1/19

(52) U.S. Cl. CPC C12P 7/62; C12N 1/20; C12N 9/0006; C12N 11/00; C12N 15/52; C12N 15/70; C12P 7/02; C12P 13/02; C12Y 101/01; C12R 2001/19

(57) ABSTRACT

The present disclosure provides an engineered ketoreductase polypeptide capable of preparing chiral alcohols, especially for catalyzing the reduction of ethyl 4-chloroacetoacetate to produce ethyl (R)-4-chloro-3-hydroxybutyrate. The polypeptides has high catalytic activity, high stereoselectivity, better thermal stability and solvent tolerance; the reaction process using engineered ketoreductase polypeptides may have a substrate loading of up to 426 g/L and a conversion of up to 99%.

### RELATED APPLICATIONS

This application is a National Phase of PCT Patent Application No. PCT/CN2023/094905 having International filing date of May 17, 2023, which claims the benefit of priority of Chinese Patent Application No. 202210753932.8 filed on Jun. 29, 2022. The contents of the above applications are all incorporated by reference as if fully set forth herein in their entirety.

### SEQUENCE LISTING STATEMENT

The XML file, entitled 101909ReplacementSequenceListing.xml, created on Nov. 3, 2025, comprising 199,885 bytes, is incorporated herein by reference. The sequence listing submitted herewith is identical to the sequence listing forming part of the international application.

### FIELD AND BACKGROUND OF THE INVENTION

The present invention relates to the field of bioengineering technology, and specifically relates to an engineered ketoreductase polypeptide and its application in the preparation of chiral alcohols.

Chiral alcohols are important compounds with a wide range of applications. Many natural compounds with biological activity contain the structure of chiral alcohols. L-carnitine, also known as vitamin BT, is an amino acid that promotes the conversion of fat into energy and has various physiological functions such as fat oxidation and decomposition, weight loss and anti-fatigue, etc. It is widely used as a food additive in infant food, diet food, athletes' food, pharmaceuticals, nutritional supplements for middle-aged and elderly people, nutritional fortification for vegetarians and animal feed additives. The chiral alcohol compound ethyl (R)-4-chloro-3-hydroxybutyrate is a key intermediate in the synthesis of L-carnitine. Therefore, the study of the efficient preparation of ethyl (R)-4-chloro-3-hydroxybutyrate has important application value and wide market prospect.

The main methods for the preparation of chiral alcohols in industrial production are chemical and enzyme-catalyzed methods. In enzyme-catalyzed methods, chiral alcohols are usually obtained by asymmetric reduction of carbonyl substrates by keto reductase (KRED) or alcohol dehydrogenase (ADH). Enzymatic methods are often favored in the industry for their high selectivity, high conversion rate, mild reaction conditions, lower cost and less pollution. In the reaction mechanism, ketoreductase or alcohol dehydrogenase catalyzes the reduction of carbonyl (or keto) compounds to alcohols via the reducing power provided by the cofactor NADH or NADPH, while the cofactor NADH or NADPH is converted to NAD+ or NADP+, respectively (FIG. 1). The production cost could be high due to the expensive cofactors NADH or NADPH, therefore, the more cost-effective cofactor recycle system is now commonly used in industry. That is, while this reaction process is going on, it is also possible to regenerate NADH or NADPH in situ by converting NAD+ or NADP+ to NADH or NADPH, respectively, through another enzymatic reaction. There are usually three enzymatic ways to achieve the conversion of NAD+ or NADP+ to NADH or NADPH, respectively. The first one is to add both glucose dehydrogenase (GDH) and glucose to the reaction in FIG. 1, in which glucose dehydrogenase catalyzes the conversion of glucose to gluconic acid, while converting NAD+ or NADP+ to NADH or NADPH, respectively; the second is to add both formate dehydrogenase (FDH) and formic acid to the reaction in FIG. 1, and convert NAD+ or NADP+ to NADH or NADPH, respectively, during the conversion of formic acid to carbon dioxide catalyzed by formate dehydrogenase; the third is to add isopropanol to the reaction in FIG. 1, and in the process of the same ketoreductase or alcohol dehydrogenase converting isopropanol to acetone, NAD+ or NADP+ is reduced to NADH or NADPH, respectively, the reaction is shown in FIG. 2.

Due to the use of additional enzymes in the GDH recycle system and the FDH recycle system, and the production costs of the first and second recycle systems mentioned above are clearly higher than those of the third system which uses only one enzyme. In addition, the application of the GDH recycle system leads to the generation of solid waste. Therefore, for cost reasons and to avoid the generation of large amounts of solid waste, the isopropanol-acetone recycle system was used in the present invention.

Ethyl (R)-4-chloro-3-hydroxybutyrate is an important chiral alcohol compound and a key intermediate of L-carnitine, which is usually obtained by chemical synthesis using metal complexes as catalysts to reduce ethyl 4-chloroacetoacetate to obtain L-carnitine IM. However, the harsh reaction conditions and the use of metal catalysts also make chemical methods costly and are accompanied by cumbersome subsequent treatment processes.

Compared to chemical methods, enzymatic processes are more economical and environmentally friendly with mild reaction conditions. Although some progress has been made in the biocatalytic synthesis of ethyl (R)-4-chloro-3-hydroxybutyrate, the reaction is not easy to control and the problem of “three wastes” still exists, for example, the application of the GDH recycle system will lead to the generation of solid waste. Therefore, in order to avoid the generation of large amounts of solid waste, the isopropanol-acetone cycle system was used in the present invention. Meanwhile, the enzyme used in the isopropanol-acetone recycle system is the same enzyme that catalyzes the reduction of ethyl 4-chloroacetoacetate to ethyl (R)-4-chloro-3-hydroxybutyrate, the production cost is also lower than that of the GDH recycle system. However, no examples of using the isopropanol-acetone recycle system to synthesize ethyl (R)-4-chloro-3-hydroxybutyrate have been reported in the prior art, and in fact, there are some challenges in using the isopropanol-acetone recycle system for synthetic reactions. Typically, enzyme-catalyzed reactions are carried out in aqueous phase systems because protein molecules are usually more stable in aqueous phase systems and most enzymes, especially wild-type enzymes that are found in nature, are unable to survive in the harsh organic phase solvent environment. However, the substrate ethyl 4-chloroacetoacetate used in the present invention is unstable in the aqueous phase system and is prone to hydrolysis, and the presence of isopropyl alcohol will further aggravate the hydrolysis of the substrate. In order to avoid spontaneous hydrolysis reaction, the reaction system in the present application uses the water-toluene two-phase system. In the case of using a water-toluene two-phase system, the enzyme disclosed in the prior art is unable to maintain sufficient activity and stability to efficiently catalyze the reduction of ethyl 4-chloroacetoacetate to ethyl (R)-4-chloro-3-hydroxybutyrate and simultaneously regenerate NADH by converting isopropanol to acetone. Therefore, in the present application, a wild-type ketoreductase has undergone directed evolution to obtain a series of engineered ketoreductase catalysts with high activity and stability in two-phase systems and tolerating higher substrate concentrations. Therefore, enzyme catalysts and reaction processes for the preparation of ethyl (R)-4-chloro-3-hydroxybutyrate with high conversion rates are obtained on the basis of achieving low cost in the present application.

The two-phase system used in the present invention not only avoids the occurrence of spontaneous hydrolysis reaction and emulsification of the substrate, but also makes it easier to control the pH of the aqueous phase during the reaction process, which is conducive to large-scale production. At the same time, the organic solvent can be recycled, which reduces the generation of wastewater and is also more environmentally friendly. The full-aqueous reaction system commonly used in the prior art produces more industrial wastewater, and organic solvent extraction is also inevitable in the subsequent purification of the product. The two-phase reaction system of the present invention reduces the amount of water used and thus significantly reduces the generation of wastewater, and it avoids the additional organic reagent extraction step, which is more in line with the requirements of green manufacturing processes and sustainable development.

In addition, since the ketoreductase reported in prior art that can catalyze the reduction of ethyl 4-chloroacetoacetate to ethyl (R)-4-chloro-3-hydroxybutyrate is unstable in the two-phase system, the use of the two-phase system with the ketoreductase reported in prior art will lead to a decrease in the conversion or total yield of the enzymatic reaction; the enzyme disclosed in this application, engineered by directed evolution, maintains high activity and stability in the two-phase system, and enables high substrate loading and high conversion (>99%) in the reaction process, and the product ee value is more than 99.8%. In addition, the engineered enzyme of this application has high thermal stability, which further meets the needs of industrial applications.

### SUMMARY OF THE INVENTION

### 1 Overview

The present invention provides an engineered ketoreductase polypeptide with high stereoselectivity, high catalytic activity, good stability and high substrate tolerance that can be used for the preparation of chiral alcohols, in particular the asymmetric synthesis of ethyl (R)-4-chloro-3-hydroxybutyrate. Also provided are genes for engineered ketoreductase polypeptides, recombinant expression vectors containing the genes, engineered strains and efficient methods for their preparation, and reaction processes for the preparation of chiral alcohols using engineered ketoreductase polypeptides.

In some embodiments, the engineered ketoreductase polypeptides disclosed in the present application are capable of catalyzing the reduction of ethyl 4-chloroacetoacetate to ethyl (R)-4-chloro-3-hydroxybutyrate with an activity greater than SEQ ID No: 2. The engineered ketoreductase polypeptide provided by the present invention has higher activity, stability, and solvent & substrate tolerance than the wild-type ketoreductase of SEQ ID NO: 2, and is able to catalyze the asymmetric reduction of ethyl 4-chloroacetoacetate to ethyl (R)-4-chloro-3-hydroxybutyrate more efficiently; or at high substrate loadings, the engineered ketoreductase polypeptide provided by the present invention are still capable of catalyzing the reduction of ethyl 4-chloroacetoacetate to ethyl (R)-4-chloro-3-hydroxybutyrate without inhibition by high substrate content. These engineered ketoreductase polypeptides may comprise amino acid sequences that differ from the sequence of SEQ ID NO: 2 in one or more residue positions selected from: X18, X22, X42, X46, X47, X48, X53, X67, X79, X80, X94, X96, X111, X115, X149, X204, X211 X229, X238, X239, X262, X282, X283, X284, X286, X303, X327. The engineered ketoreductase polypeptide comprises an amino acid sequence having at least one of the following features: V18I, A22K, I42L, D46G, D46F, D46V, M47I, M47S, P48R, P48H, I53Q, I53G, I53P, I53T, I53S, I53H, I53N, I53R, I53K, T67V, T67I, E79K, T80V, A94R, A94Y, A94C, A94H, H96R, E111H, T115R, T115C, T115S, T115H, V149A, L204I, L211F, D229A, E238R, E238K, G239S, I262V, F282Q, F282G, M283N, M283D, I284M, I284L, I284V, F286V, F286H, F286R, D303E, T327L, T327E; or in addition to the abovementioned differences, the engineered ketoreductase polypeptides comprise insertions or deletions of one or more amino acid residues.

More specifically, in some embodiments, the engineered ketoreductase polypeptide improved upon SEQ ID NO: 2 comprises polypeptides consisting of the amino acid sequences corresponding to SEQ ID NO: 4, 6, 8, 10, 12, 14, 16, 18, 20, 22, 24, 26, 28, 30, 32, 34, 36, 38, 40, 42, 44, 46, 48, 50, 52, 54, 56 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128.

In some embodiments, the improved engineered ketoreductase polypeptide comprises amino acid sequences having at least 80%, 85%, 86%, 87%, 88 89%, 90%, 91%, 92%, 93%, 94%, 95%, 96%, 97%, 98%, 99% or more sequence identity to the reference sequence of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 72, 74, 76, 78, 80, 82, 84, 86, 88, 90, 92, 94, 96, 98, 100, 102, 104, 106, 108, 110, 112, 114, 116, 118, 120, 122, 124, 126, 128. In some embodiments, the engineered ketoreductase polypeptide catalyzes the asymmetric reduction of ethyl 4-chloroacetoacetate to ethyl (R)-4-chloro-3-hydroxybutyrate, said polypeptide comprising an amino acid sequence having at least 90% sequence identity to the reference sequence SEQ ID NO:2 and having at least two residue differences at residue positions X204, X211 compared to SEQ ID NO:2, wherein the amino acid residue at position X204 is selected from I and the amino acid residue at position X211 is selected from F. Said polypeptide has better activity, stability and solvent & substrate tolerance than SEQ ID NO:2, and gives a product ee value of at least 99%.

The identity between two amino acid sequences or two nucleotide sequences can be obtained by algorithms commonly used in the art, either by using the NCBI Blastp and Blastn software based on default parameters, or by using the Clustal W algorithm (Nucleic Acid Research, 22 (22): 4673-4680, 1994).

In another aspect, the present invention provides polynucleotide sequences encoding engineered ketoreductase polypeptides. In some embodiments, the polynucleotide may be a portion of an expression vector having one or more control sequences for expression of the engineered ketoreductase polypeptide. In some embodiments, the polynucleotide may comprise polynucleotide sequence corresponding to SEQ ID No: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73 75, 77, 79, 81, 83, 85, 87, 89, 91, 93, 95, 97, 99, 101, 103, 105, 107, 109, 111, 113, 115, 117, 119, 121, 123, 125, 127.

As known to those of skill in the art, due to the degeneracy of the nucleotide codons, the polynucleotide sequences encoding the amino acid sequences of SEQ ID No: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128 are not limited to SEQ ID No: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89 91, 93, 95, 97, 99, 101, 103, 105, 107, 109, 111, 113, 115, 117, 119, 121, 123, 125, 127. The nucleic acid sequence encoding the ketoreductase of the present invention may also be any other nucleic acid sequence encoding the amino acid sequence shown in SEQ ID No: 4, 6, 8, 10, 12, 14, 16, 18, 20, 22, 22, 26, 28, 30, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128.

In another aspect, the present disclosure provides expression vectors and host cells comprising a polynucleotide encoding an engineered ketoreductase or capable of expressing an engineered ketoreductase. In some embodiments, the host cell may be a bacterial host cell, such as E. coli. The host cell can be used to express and isolate the engineered ketoreductase described herein, or optionally used directly to reactively transform the substrate into a product.

In some embodiments, the engineered ketoreductase in the form of intact cells, crude extracts, isolated polypeptides, or purified polypeptides may be used alone, or in an immobilized form (e.g., immobilized on a resin).

In another aspect, the improved engineered ketoreductase polypeptide described herein converts carbonyl compounds to chiral alcohols in the presence of the cofactor NADH. The present disclosure also provides methods for preparing a broad range of compounds (I) or structural analogs thereof using the engineered ketoreductase polypeptides disclosed herein. In some embodiments, the engineered ketoreductase polypeptides may be used in methods for preparing compounds of structural formula (I):

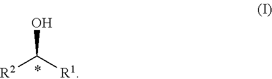

The alcohol product of said structural formula (I) has the indicated stereochemical configuration shown at the chiral center marked with an *; the alcohol product of said structure (I) is in enantiomeric excess over the other isomer, where

R¹ is optionally substituted or unsubstituted aryl or heteroaryl, or optionally substituted or unsubstituted C₁-C₅ hydrocarbon group, or may be a cyclic hydrocarbon group or a heterocyclic group.

R² is an optionally substituted or unsubstituted C₁-C₆ hydrocarbon group, a halogen (such as —F, —Cl, —Br and —I), an alkenyl, an alkynyl, an aryl, a heteroaryl, —NO₂, —NO, —SO₂R′ or —SOR′, —SR′, —NR′R′, —OR′, —CO₂R′ or —COR′, —C(O)NR′, —SO₂NH₂ or —SONH₂, —CN, —CF₃; wherein each R′ is independently selected from —H, C₁-C₄ hydrocarbon group, halogen, C₁-C₈ hydrocarbon group, C₂-C₁₂ alkenyl group, C₂-C₁₂ alkynyl group, cyclic hydrocarbon group, aryl group or heterocyclic group.

The process comprises contacting a carbonyl substrate of structural formula (II)

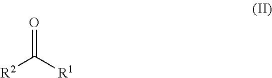

with a ketoreductase polypeptide under reaction conditions suitable for converting the carbonyl substrate to an alcohol product, wherein said ketoreductase polypeptide is a ketoreductase polypeptide as described herein comprising the amino acid sequences corresponding to SEQ ID No: 4, 6, 8, 10, 12, 14, 16, 18, 20, 22, 24, 26, 28, 30, 32, 34, 36, 38, 40, 40, 42, 44, 46, 48, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118 120, 122, 124, 126, 128.

### 2.1 Definition

With respect to this disclosure, unless otherwise expressly defined, the technical terms and scientific terms used in the specification herein have the meanings commonly understood by those of ordinary skill in the art.

The terms “protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristoylation, ubiquitination, etc.). The definition includes D-amino acids and L-amino acids, as well as mixtures of D-amino acids and L-amino acids.

“Engineered ketoreductase”, “engineered ketoreductase polypeptide”, “improved ketoreductase polypeptide”, and “engineered polypeptide” are used interchangeably herein.

Ketoreductase or alcohol dehydrogenase are used interchangeably herein.

The terms “polynucleotide” and “nucleic acid” are used interchangeably herein.

“Cofactor” as used herein refers to a non-protein compound that operates in conjunction with an enzyme in a catalytic reaction. As used herein, “cofactors” include NADH (nicotinamide adenine dinucleotide) or NADPH (nicotinamide adenine dinucleotide phosphate) and their oxidized forms NAD+ or NADP+, which are sometimes also referred to as coenzymes.

“Coding sequence” refers to that portion of a nucleic acid (e.g., a gene) that encodes an amino acid sequence of a protein.

“Naturally occurring” or “wild-type” refers to the form found in nature. For example, a naturally-occurring or wild-type polypeptide or polynucleotide sequence is a sequence that is present in an organism that can be isolated from sources in nature and which has not been intentionally modified by manual procedures.

“Recombinant” or “engineered” or “non-naturally occurring” when used with reference to, for example, a cell, nucleic acid or polypeptide, refers to a material or material corresponding to the native or native form of the material, that has been modified in a manner that would not otherwise exist in nature, or is identical thereto but produced or derived from synthetic material and/or by manipulation using recombinant techniques.

The terms “sequence identity” and “homology” are used interchangeably herein to refer to comparisons between polynucleotide sequences or polypeptide sequences (“sequence identity” or “homology” is generally expressed as a percentage), and are determined by comparing two optimally aligned sequences over a comparison window, where the portion of the polynucleotide or polypeptide sequence in the comparison window may comprise additions or deletions (i.e., gaps) as compared to the reference sequence for optimal alignment of the two sequences. The percentage may be calculated by determining the number of positions at which either the identical nucleic acid bases or amino acid residue is aligned with a gap to yield the number of matched positions, dividing the number of matched positions by the total number of positions in the window of comparison and multiplying the result by 100 to yield the percentage of sequence identity. Optionally, the percentage may be calculated by determining the number of positions where the same nucleic acid base or amino acid residue is present in both sequences or the number of positions where the nucleic acid base or amino acid residue is aligned with a null position to obtain the number of matching positions, dividing that number of matching positions by the total number of positions in the comparison window, and multiplying the result by 100 to obtain the percentage of sequence identity. Those of skill in the art will appreciate that there are many established algorithms available to align two sequences. The optimal alignment of sequences for comparison can be conducted, for example, by the local homology algorithm of Smith and Waterman, 1981, Adv. Appl. Math. 2:482, by the Homology alignment algorithm of Needleman and Wunsch, 1970, J. Mol. Biol. 48:443, by the homology comparison algorithm of Pearson and Lipman, 1988, Proc. Natl. Acad. Sci. USA85:2444, by computerized implementations of these algorithms (GAP, BESTFIT, FASTA, or TFASTA in the GCG Wisconsin package) or by visual inspection (see generally, Current Protocols in Molecular Biology, edited by F. M. Ausubel et al, Current Protocols, a joint venture between Greene Publishing Associates Inc. and John Wiley & Sons, Inc. (1995 supplement) (Ausubel)). Examples of algorithms that are suitable for determining the percent sequence identity and percent sequence similarity are the BLAST and BLAST 2.0 algorithms, which are described in Altschul et al, 1990, J. Mol. Biol. 215:403-410 and Altschul et al, 1977, Nucleic Acids Res. 3389-3402, respectively. Software for performing BLAST analysis is publicly available through the National Center for Biotechnology Information (NCBI) website. The algorithm involves first identifying high scoring sequence pairs (HSPs) by identifying short words of length W in the query sequence, which either match or satisfy some positive-valued threshold scores T when aligned with a word of the same length in the database sequence. T is referred to as, the neighborhood word score threshold (Altschul et al., as described above). These initial neighborhood word hits serve as seeds for initiating searches to find longer HSPs that contain them. The word hits are then extended in both directions along each sequence for as far as the cumulative alignment score can be increased. For nucleotide sequences, the cumulative score is calculated using the parameters M (reward score for matched pair of residues; always >0) and N (penalty score for mismatched residues; always <0). For amino acid sequences, a scoring matrix is used to calculate the cumulative score. The extension of the word hits in each direction are halted when: the cumulative alignment score falls off by the quality X from its maximum achieved value; the cumulative score goes 0 or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, the expected value (E) of 10, M=5, N=−4, and a comparison of both strands as a default value. For amino acid sequences, the BLASTP program uses as defaults the wordlength (W) of 3, the expected value (E) of 10 and the BLOSUM62 scoring matrix (see Henikoff and Henikoff, 1989, Proc Natl Acad Sci USA 89: 10915). Exemplary determination of sequence alignments and % sequence identity can employ the BESTFIT or GAP programs in the GCG Wisconsin Software package (Accelrys, Madison WI), using the default parameters provided.

“Reference sequence” refers to a defined sequence that is used as a basis for sequence comparison. The reference sequence may be a subset of a larger sequence, for example, a full-length gene or a fragment of a polypeptide sequence. In general, a reference sequences is at least 20 nucleotides or amino acid residues in length, at least 25 residues long, at least 50 residues in length, or the full length of a nucleic acid or polypeptide. Because two polynucleotides or polypeptides may each (1) comprise a sequence (i.e., a portion of the complete sequence) that is similar between the two sequences, and (2) may further comprise sequences that is divergent between the two sequences, sequence comparisons between two (or more) polynucleotides or polypeptides are typically performed by comparing the sequences of the two (or more) polynucleotides or polypeptides over a “comparison window” to identify and compare local regions of sequence similarity. In some embodiments, a “reference sequence” is not intended to be limited to wild-type sequences, and may comprise engineered or altered sequences. For example, “a reference sequence with proline at the residue corresponding to X53 based on SEQ ID NO: 2” refers to a reference sequence wherein the corresponding residue (being an isoleucine) at X53 in SEQ ID NO:2 has been altered to a proline.

“Comparison window” refers to a conceptual segment of at least about 20 contiguous nucleotide positions or amino acid residues, wherein the sequence may be compared to a reference sequence of at least 20 contiguous nucleotides or amino acids and wherein the portions of the sequence in the comparison window may comprise additions or deletions (i.e., gaps) of 20% or less as compared to a reference sequence (which does not comprise additions or deletions) for optimal alignment of the two sequences. The comparison window can be longer than 20 contiguous residues, and optionally include 30, 40, 50, 100 or more residues.

In the context of the numbering for a given amino acid or polynucleotide sequence, “corresponding to”, “reference to” or “relative to” refers to the numbering of the residues of a specified reference when the given amino acid or polynucleotide sequence is compared to the reference sequence. In other words, the residue number or residue position of a given sequence is designated with respect to the reference sequence, rather than by the actual numerical position of the residue within the given amino acid or polynucleotide sequence. For example, a given amino acid sequence such as an engineered ketoreductase can be aligned to a reference sequence by introducing gaps to optimize residue matches between the two sequences. In these cases, although there are gaps, the numbering of the residue in a given amino acid or polynucleotide sequence is made with respect to the reference sequence to which they have been aligned.

An “amino acid difference” or “residue difference” refers to the difference in amino acid residues at a position of a polypeptide sequence relative to the amino acid residue at a corresponding position in a reference sequence. The position of an amino acid difference is generally referred to herein as “Xn”, where n refers to the corresponding position in the reference sequence on which the residue difference is based. For example, “a residue difference at position X53 as compared to SEQ ID NO:2” refers to the difference in amino acid residues at the polypeptide position corresponding to position 53 of SEQ ID NO:2. Thus, if the reference polypeptide of SEQ ID NO:2 has an isoleucine at position 53, then “a residue difference at position X53 as compared to SEQ ID NO:2” refers to an amino acid substitution of any residue other than isoleucine at the position of the polypeptide corresponding to position 53 of SEQ ID NO:2. In most of the examples herein, the specific amino acid residue difference at the position is indicated as “XnY”, wherein “Xn” specified to the corresponding position as described above, and “Y” is the single letter identifier of the amino acid found in the engineered polypeptide (i.e., a different residue than in the reference polypeptide). In some examples (e.g., in Table 1), the present disclosure also provides specific amino acid differences denoted by the conventional notation “AnB”, where A is a single letter identifier of a residue in the reference sequence, “n” is the number of residue position in the reference sequence, and B is the single letter identifier for the residue substitution in the sequence of the engineered polypeptide. In some examples, an engineered polypeptide of this disclosure may comprise one or more amino acid residue differences relative to a reference sequence, which is indicated by a list of specific positions at which residue differences are present relative to a reference sequence.

“Deletion” refers to the modification of a polypeptide by removing one or more amino acids from a reference polypeptide. Deletions may include removal of one or more, two or more amino acids, five or more amino acids, ten or more amino acids, fifteen or more amino acids, or twenty or more amino acids, up to 10% of the total number of amino acids of the enzyme, or up to 20% of the total number of amino acids making up the reference enzyme while retaining the enzymatic activity of the engineered ketoreductase and/or retaining the improved properties of the engineered ketoreductase. Deletion may involve the internal portion and/or the terminal portion of the polypeptide. In various embodiments, deletions may include a contiguous segment or may be discontinuous, and/or retaining the improved properties of the engineered ketoreductase.

“Insertion” refers to the modification of a polypeptide by adding one or more amino acids from a reference polypeptide. In some embodiments, the improved engineered ketoreductase comprises insertions of one or more amino acids to a naturally-occurring ketoreductase polypeptide, as well as insertions of one or more amino acids to other engineered ketoreductase polypeptides. It can be inserted in the internal portions of the polypeptide or inserted to the carboxyl or amino terminus. As used herein, insertions include fusion proteins known in the art. The insertion may be a contiguous segment of amino acids or separated by one or more amino acids in naturally occurring or engineered polypeptide.

“Fragment” as used herein refers to a polypeptide having an amino terminal and/or carboxy terminal deletion, but where the remaining amino acid sequence is identical to the corresponding position in the sequence. Fragments may be at least 10 amino acids long, at least 20 amino acids long, at least 50 amino acids long or longer, and up to 70%, 80%, 90%, 95%, 98% and 99% of the full-length ketoreductase polypeptide.

The term “isolated polypeptide” refers to a polypeptide that is substantially separated from other substances with which it is naturally associated, such as proteins, lipids, and polynucleotides. The term comprises polypeptides that have been removed or purified from their naturally occurring environment or expression system (e.g., in host cells or in vitro synthesis). Engineered ketoreductase polypeptides may be present in the cell, in the cell culture medium, or prepared in various forms, such as lysates or isolated preparations. As such, in some embodiments, the engineered ketoreductase polypeptide may be an isolated polypeptide.

“Chiral center” refers to a carbon atom connecting four different groups.

“Stereoselectivity” refers to the preferential formation of one stereoisomer over the other in a chemical or enzymatic reaction. Stereoselectivity can be partial, with the formation of one stereoisomer is favored over the other, or it may be complete where only one stereoisomer is formed. When the stereoisomers are enantiomers, the stereoselectivity is referred to as enantioselectivity, and the excess fraction (usually reported as a percentage) of an enantiomer in a mixture of two enantiomers is optionally reported as “enantiomeric excess” (ee for short). When the stereoisomers are diastereoisomers, the stereoselectivity is referred to as diastereoselectivity, i.e. the excess fraction (usually reported as a percentage) of a diastereoisomer in a mixture of two diastereoisomers, which is optionally reported as “diastereoisomeric excess” (de for short). This fraction (typically a percentage) is optionally reported in the art as an enantiomeric excess (ee) calculated from the following equation: {major enantiomer concentration−minor enantiomer concentration}/{major enantiomer concentration+minor enantiomer concentration}.

The terms “stereoisomers”, “stereoisomeric forms” and similar expressions are used interchangeably herein to refer to all isomers resulting from a difference in orientation of atoms in their space only. It includes enantiomers and compounds that have more than one chiral center and are not mirror images of one another (i.e., diastereomers).

“Improved enzymatic properties” refers to an enzyme property that is better or more desirable for a specific purpose as compared to a reference ketoreductase such as a wild-type ketoreductase or another improved engineered ketoreductase. Improved enzyme properties include, but are not limited to, enzyme activity (which can be expressed as a percentage conversion of substrate), thermal stability, solvent stability, pH activity characteristics, cofactor requirements, tolerance to inhibitors (e.g., substrate or product inhibition), stereospecificity, and stereoselectivity.

“Conversion” refers to the enzymatic transformation of the substrate to the corresponding product. “Percent conversion” or “conversion” refers to the percentage of substrate that is converted to a product within a period of time under the specified reaction conditions. Thus, “enzymatic activity” or “activity” of a ketoreductase polypeptide can be expressed as the “percent conversion” of the substrate to the product. The conversion is generally calculated by taking a sample to determine the product concentration and substrate concentration in the reaction system: {molar concentration of product}/{molar concentration of substrate+molar concentration of product}.

“Thermalstable” means that a ketoreductase polypeptide that retains similar activity after exposure to elevated temperatures (e.g., 68° C. or higher) for a period of time (e.g., 2.5 hours or longer).

“Solvent stable” or “solvent tolerant” means that the ketoreductase polypeptide maintains similar activity after exposure to varying concentrations (e.g., 5-99%) of solvents (methanol, ethanol, isopropanol, dimethyl sulfoxide (DMSO), tetrahydrofuran, 2-methyl tetrahydrofuran, acetone, toluene, butyl acetate, methyl tert-butyl ether, etc.) for a period of time (e.g., 0.5-24 h).

“Suitable reaction conditions” refers to those conditions (e.g., range of enzyme loading, substrate loading, cofactor loading, temperature, pH, buffer, co-solvent, etc.) in the biocatalytic reaction system, including also the reactant material for cofactor cycling regeneration, under which the ketoreductase polypeptide of the present disclosure converts the substrate to the desired product compound. Exemplary “suitable reaction conditions” are provided in the present disclosure and illustrated by examples.

“Hydrocarbyl” or “hydrocarbon group” refers to a straight or branched hydrocarbon chain. The number of subscripts following the symbol “C” specifies the number of carbon atoms that a particular chain may contain. For example, “C₁-C₈” refers to a straight or branched hydrocarbon group having 1 to 8 carbon atoms. Hydrocarbyl groups may optionally be substituted with one or more substituent groups. “Aryl” means a monovalent aromatic hydrocarbon group of 6 to about 20 carbon atoms. “Heteroaryl” and “heteroaromatic” refer to aryl groups in which one or more of the carbon atoms of the parent aromatic ring system is/are replaced by heteroatom (O, N or S). “Substituted” when used to modify a designated group or radical means that one or more hydrogen atoms of the designated group or radical are each replaced, independently of one another, by identical or different substituents. “Substituted hydrocarbyl, aryl or heteroaryl” refers to a hydrocarbyl, aryl, or heteroaryl group in which one or more hydrogen atoms are replaced by other substituents. “Optional” or “optionally” means that the described event or circumstance may or may not occur; for example, “optionally substituted aryl” refers to an aryl group that may or may not be substituted. The description includes both substituted aryl groups and unsubstituted aryl groups.

The term “compound” as used herein refers to any compound encompassed by the structural formula and/or chemical name indicated with a compound disclosed herein. A compound may be identified by its chemical structure and/or chemical name. Where the chemical structure and the chemical name conflict, the chemical structure determines the identity of the compound. Unless specifically stated or indicated otherwise, the chemical structures described herein encompass all possible isomeric forms of the described compounds.

### 2.2 Improved Engineered Ketoreductases

Table 1 below provides examples of the engineered ketoreductase polypeptides developed by the present invention. Each row gives the polynucleotide sequence number and amino acid sequence number of a specific engineered ketoreductase polypeptide, as well as the residue differences compared to SEQ ID No: 2. The level of catalytic performance of each exemplified engineered ketoreductase polypeptide under different reaction conditions are shown in Table 1.

TABLE 1

| | SEQ ID | | Catalytic performance | | | |
|---|---|---|---|---|---|---|
| SEQ ID | NO | | fold (relative to SEQ ID | | | |
| NO | (Amino | Amino acid residue differences relative | NO: 2) | | | |
| (DNA) | acid) | to SEQ ID NO: 2 | ¹Fold | ²Fold | ³Fold | ⁴Conversion |
| 1 | 2 | — | 1 | 1 | 1 | 3.20% |
| 3 | 4 | I42L; L204I; L211F; F282Q; M283N | 3.36 | 8.13 | 17.56 | 96.50% |
| 5 | 6 | V18I; L204I; L211F; F282Q; M283N | 3.84 | 8.65 | 18.93 | 97.53% |
| 7 | 8 | E79K; L204I; L211F; F282Q; M283N | 4.23 | 9.12 | 19.86 | 98.45% |
| 9 | 10 | I53Q; L204I; L211F; F282Q; M283N | 3.64 | 8.36 | 18.45 | 97.17% |
| 11 | 12 | I53G; L204I; L211F; F282Q; M283N | 4.76 | 9.59 | 21.04 | 98.85% |
| 13 | 14 | A22K; L204I; L211F; F282Q; M283N | 5.24 | 10.32 | 22.26 | 99.42% |
| 15 | 16 | I53P; L204I; L211F; F282Q; M283N | 6.24 | 11.52 | 24.63 | 99.45% |
| 17 | 18 | I53T; L204I; L211F; F282Q; M283N | 5.35 | 10.63 | 22.35 | 99.73% |
| 19 | 20 | I53S; L204I; L211F; F282Q; M283N | 6.06 | 11.10 | 24.84 | 99.64% |
| 21 | 22 | L204I; L211F; E238R; F282Q; M283N | 4.68 | 9.28 | 21.45 | 98.85% |
| 23 | 24 | L204I; L211F; E238K; F282Q; M283N | 5.12 | 10.41 | 22.77 | 99.86% |
| 25 | 26 | L204I; L211F; F282Q; M283N; T327L | 4.63 | 9.35 | 21.57 | 98.75% |
| 27 | 28 | L204I; L211F; F282Q; M283N; T327E | 3.74 | 8.71 | 18.64 | 96.45% |
| 29 | 30 | E111H; L204I; L211F; F282Q; M283N | 3.36 | 8.34 | 18.45 | 96.13% |
| 31 | 32 | T115R; L204I; L211F; F282Q; M283N | 4.68 | 9.51 | 21.87 | 98.76% |
| 33 | 34 | T115C; L204I; L211F; F282Q; M283N | 4.07 | 9.02 | 20.55 | 98.45% |
| 35 | 36 | T115S; L204I; L211F; F282Q; M283N | 3.84 | 8.66 | 20.76 | 97.41% |
| 37 | 38 | T115H; L204I; L211F; F282Q; M283N | 4.86 | 9.63 | 22.94 | 98.93% |
| 39 | 40 | L204I; L211F; F282G; M283D | 2.35 | 7.22 | 15.21 | 95.51% |
| 41 | 42 | T80V; L204I; L211F; D229A; I262V; | 6.92 | 11.67 | 25.02 | 99.60% |
| | | F282Q; M283N; D303E | | | | |
| 43 | 44 | T80V; V149A; L204I; L211F; D229A; | 6.75 | 11.54 | 25.04 | 99.71% |
| | | I262V; F282Q; M283N | | | | |
| 45 | 46 | T80V; L204I; L211F; I262V; F282Q; | 5.24 | 10.59 | 22.85 | 99.31% |
| | | M283N | | | | |
| 47 | 48 | T80V; V149A; L204I; L211F; D229A; | 6.39 | 11.48 | 24.45 | 99.34% |
| | | F282Q; M283N | | | | |
| 49 | 50 | T80V; L204I; L211F; D229A; F282Q; | 5.4 | 10.93 | 23.75 | 98.36% |
| | | M283N | | | | |
| 51 | 52 | T80V; L204I; L211F; F282Q; M283N | 5.3 | 10.68 | 23.56 | 98.24% |
| 53 | 54 | T80V; V149A; L204I; L211F; F282Q; | 5.6 | 11.02 | 23.89 | 99.46% |
| | | M283N | | | | |
| 55 | 56 | T80V; L204I; L211F; D229A; I262V; | 5.45 | 10.86 | 23.67 | 99.44% |
| | | F282Q; M283N | | | | |
| 57 | 58 | T80V; V149A; L204I; L211F; D229A; | 6.54 | 11.85 | 24.87 | 99.91% |
| | | F282Q; M283N; D303E | | | | |
| 59 | 60 | T80V; L204I; L211F; F282Q; M283N; | 7.25 | 11.83 | 25.45 | 99.96% |
| | | D303E | | | | |
| 61 | 62 | T80V; L204I; L211F; I262V; F282Q; | 6.44 | 11.24 | 24.87 | 99.60% |
| | | M283N; D303E | | | | |
| 63 | 64 | I53P; L204I; L211F; F282Q; M283N; | 7.2 | 11.69 | 24.99 | 99.83% |
| | | I284M; F286V | | | | |
| 65 | 66 | I53P; L204I; L211F; F282Q; M283N; | 6.57 | 11.36 | 24.34 | 99.62% |
| | | F286H | | | | |
| 67 | 68 | I53H; L204I; L211F; F282Q; M283N; | 6 | 11.05 | 23.97 | 99.44% |
| | | F286V | | | | |
| 69 | 70 | I53P; L204I; L211F; F282Q; M283N; | 6.95 | 11.67 | 24.69 | 99.82% |
| | | F286V | | | | |
| 71 | 72 | I53P; L204I; L211F; F282Q; M283N; | 8.5 | 11.98 | 25.93 | 99.97% |
| | | F286R | | | | |
| 73 | 74 | I53N; L204I; L211F; F282Q; M283N; | 5.93 | 10.52 | 24.50 | 99.61% |
| | | I284M; F286V | | | | |
| 75 | 76 | I53H; L204I; L211F; F282Q; M283N; | 6 | 11.01 | 24.68 | 99.70% |
| | | F286R | | | | |
| 77 | 78 | I53T; L204I; L211F; F282Q; M283N; | 5.32 | 10.13 | 22.97 | 99.51% |
| | | I284L; F286V | | | | |
| 79 | 80 | I53N; L204I; L211F; F282Q; M283N; | 6.41 | 11.42 | 24.84 | 99.76% |
| | | F286H | | | | |
| 81 | 82 | I53P; L204I; L211F; F282Q; M283N; | 5.83 | 10.36 | 22.95 | 99.59% |
| | | I284L; F286V | | | | |
| 83 | 84 | I53P; L204I; L211F; F282Q; M283N; | 5.68 | 10.27 | 22.86 | 99.51% |
| | | I284V; F286R | | | | |
| 85 | 86 | I53T; L204I; L211F; F282Q; M283N; | 5.52 | 10.21 | 22.64 | 99.43% |
| | | I284V; F286R | | | | |
| 87 | 88 | I53R; L204I; L211F; F282Q; M283N; | 5.47 | 10.19 | 22.60 | 99.49% |
| | | F286R | | | | |
| 89 | 90 | I53P; L204I; L211F; F282Q; M283N; | 6.75 | 11.58 | 24.63 | 99.81% |
| | | I284M; F286R | | | | |
| 91 | 92 | I53T; L204I; L211F; F282Q; M283N; | 6.01 | 11.13 | 24.10 | 99.36% |
| | | F286V | | | | |
| 93 | 94 | I53P; L204I; L211F; F282Q; M283N; | 6.11 | 11.19 | 24.35 | 99.69% |
| | | I284V; F286H | | | | |
| 95 | 96 | I53K; L204I; L211F; F282Q; M283N; | 6.54 | 11.58 | 24.92 | 99.78% |
| | | F286V | | | | |
| 97 | 98 | I53P; L204I; L211F; F282Q; M283N; | 6.52 | 11.58 | 24.58 | 99.76% |
| | | I284V; F286V | | | | |
| 99 | 100 | D46G; L204I; L211F; F282Q; M283N | 2.75 | 7.32 | 16.06 | 95.19% |
| 101 | 102 | D46F; L204I; L211F; F282Q; M283N | 4.25 | 9.08 | 22.34 | 99.01% |
| 103 | 104 | D46V; L204I; L211F; F282Q; M283N | 2.85 | 7.40 | 17.06 | 95.45% |
| 105 | 106 | P48R; L204I; L211F; F282Q; M283N | 2.83 | 7.43 | 16.10 | 94.50% |
| 107 | 108 | M47I; L204I; L211F; F282Q; M283N | 2.75 | 7.29 | 15.93 | 94.39% |
| 109 | 110 | M47S; L204I; L211F; F282Q; M283N | 2.72 | 7.25 | 15.89 | 94.37% |
| 111 | 112 | P48H; L204I; L211F; F282Q; M283N | 3.21 | 8.14 | 19.17 | 96.13% |
| 113 | 114 | P48H; L204I; L211F; G239S; F282Q; | 2.7 | 7.27 | 15.34 | 94.05% |
| | | M283N | | | | |
| 115 | 116 | T67V; L204I; L211F; F282Q; M283N | 3.18 | 8.21 | 19.06 | 96.32% |
| 117 | 118 | T67I; L204I; L211F; F282Q; M283N | 4.02 | 9.87 | 21.16 | 98.03% |
| 119 | 120 | H96R; L204I; L211F; F282Q; M283N | 3.08 | 8.09 | 18.05 | 96.26% |
| 121 | 122 | A94R; L204I; L211F; F282Q; M283N | 2.98 | 7.74 | 17.83 | 95.76% |
| 123 | 124 | A94Y; L204I; L211F; F282Q; M283N | 2.98 | 7.69 | 17.16 | 96.05% |
| 125 | 126 | A94C; L204I; L211F; F282Q; M283N | 2.95 | 7.65 | 17.22 | 95.93% |
| 127 | 128 | A94H; L204I; L211F; F282Q; M283N | 2.9 | 7.66 | 16.41 | 94.84% |

Note:
Catalytic performance fold in Table 1 refers to the ratio of the conversion of Ethyl 4-chloroacetoacetate to ethyl (R)-4-chloro-3-hydroxybutyrate by the engineered ketoreductase to that by SEQ ID NO: 2 in different reaction conditions. The ee values of ethyl (R)-4-chloro-3-hydroxybutyrate obtained using the engineered ketoreductases in Table 1 were all greater than 99%.
“¹Fold” was measured under Reaction condition 1: Ethyl 4-chloroacetoacetate 180 g/L, wet cell 10 g/L, NAD+ 0.5 g/L, IPA 25% (V/V), 0.1M PB, 30° C., pH 7.0, 24 h;
“²Fold” was measured under Reaction condition 2: Ethyl 4-chloroacetoacetate 250 g/L, wet cell 10 g/L, NAD+ 0.5 g/L, IPA/toluene/water: 25%/40%/35% (V/V/V), 0.1M PB, 30° C., pH 7.0, 24 h;
“³Fold” was measured under Reaction condition 3: Ethyl 4-chloroacetoacetate 250 g/L, [68° C., 4 h] heat-treated wet cell 10 g/L, NAD+ 0.5 g/L, IPA/toluene/water: 25%/40%/35% (V/V/V), 0.1M PB, 30° C., pH 7.0, 24 h;
“⁴Conversion” was measured under Reaction condition 4: Ethyl 4-chloroacetoacetate 400 g/L, Wet cell 30 g/L, NAD+ 0.5 g/L, IPA/Toluene/Water: 25%/40%/35% (V/V/V), 0.1M PB, 30° C., pH 7.0, 24 h.

The wet cells in reaction conditions 1, 2, and 4 were prepared with reference to Example 2. The wet cell after heat treatment in reaction condition 3 was obtained by heat treatment at [68° C., 4 h] on the basis of the wet cell described above.

Under the reaction condition 1, the catalytic activity of the engineered ketoreductase disclosed in the present application is higher than that of the wild-type enzyme, reaching up to 8.5 fold of the wild-type enzyme. Under reaction condition 2, in the presence of organic solvent toluene, the engineered ketonereductase disclosed in the present application is better adapted to the two-phase system and has better solvent tolerance, and the catalytic activity of the engineered ketonereductase is about 7-12 fold of the wild-type enzyme. Under reaction condition 3, due to the difference in thermal stability of ketoreductase polypeptides listed in Table 1, after heat treatment, different ketoreductase polypeptides would exhibit different degrees of inactivation leading to changes in catalytic performance, and the catalytic activity of the engineered ketoreductase disclosed in the present application is about 15-26 fold of the wild-type enzyme; it can be seen that the engineered ketoreductase disclosed in the present application has improved thermal stability. Under reaction condition 4, the loading of substrate ethyl 4-chloroacetoacetate was increased to 400 g/L, and the conversion by SEQ ID NO: 2 was low due to the inhibitory effect of the substrate on the wild-type enzyme, while the substrate tolerance of the engineered enzyme was much better, and at the substrate loading of 400 g/L, the engineered ketoreductase disclosed in the present application could still achieve a conversion of more than 94%, up to 99.97%.

### 2.3 Polynucleotides, Control Sequences, Expression Vectors and Host Cells that can be Used to Produce Engineered Ketoreductase Polypeptides

In another aspect, the present disclosure provides polynucleotides encoding the engineered polypeptides having ketoreductase activity described herein. The polynucleotides may be operably linked to one or more heterologous regulatory sequences that control gene expression to produce a recombinant polynucleotide that are capable of expressing the polypeptide. Expression constructs comprising heterologous polynucleotides encoding engineered ketoreductases may be introduced into suitable host cells to express the corresponding engineered ketoreductase polypeptides.

As apparent to those of skill in the art, the availability of protein sequences and knowledge of codons corresponding to a variety of amino acids provides an illustration of all possible polynucleotides that encode the protein sequence of interest. The degeneracy of the genetic code, in which the same amino acids are encoded by selectable or synonymous codons, allows for the production of an extremely large number of polynucleotides, all of which encode the improved ketoreductase polypeptides disclosed herein. Thus, upon determination of a particular amino acid sequence, one of skill in the art can generate any number of different polynucleotides by merely modifying the sequence of one or more codons in a manner that does not alter the amino acid sequence of the protein. In this regard, the present disclosure particularly contemplates each and every possible alteration of polynucleotides that can be made by selecting a combination based on possible codon selections, and any of the polypeptides disclosed herein, comprising the amino acid sequences of the exemplary engineered polypeptides provided in Table 1.

In various embodiments, the codons are preferably selected to accommodate the host cell in which the recombinant protein is produced. For example, codons preferred for bacteria are used to express genes in bacteria; codons preferred for yeast are used to express genes in yeast; and codons preferred for mammals are used for gene expression in mammalian cells.

In some embodiments, the polynucleotides encode polypeptides comprising amino acid sequences that are at least about 80%, 85%, 86%, 87%, 88%, 89%, 90%, 91%, 92%, 93%, 94%, 95%, 96%, 97%, 98%, 99% or more identical to a reference sequence that is an even sequence identifier of SEQ ID NO: 4-128, wherein the polypeptides have ketoreductase activity and one or more of the improved properties described herein, such as the ability to convert a substrate to a product with increased activity compared to the polypeptide of SEQ ID NO: 2.

In some embodiments, the polynucleotides encoding the engineered ketoreductase polypeptides comprise sequences having odd sequence identifiers of SEQ ID NO:3-127.

In some embodiments, the polynucleotides encode polypeptides as described herein, but at the nucleotide level, the polynucleotide has about 50%, 55%, 60%, 65%, 70%, 75%, 80%, 85%, 86%, 87%, 88%, 89%, 90%, 91%, 92%, 93%, 94%, 95%, 96%, 97%, 98%, 99% or more sequence identity to reference polynucleotides encoding engineered ketoreductases. In some embodiments, the reference polynucleotide sequence is selected from a sequence having the odd sequence identifier of SEQ ID NO: 3-127.

The isolated polynucleotides encoding the engineered ketoreductase polypeptides can be manipulated to enable the expression of the polypeptides in a variety of ways, which comprises further modification of the sequence by codon optimization to improve expression, insertion into suitable expression elements with or without additional control sequences, and transformation into host cells suitable for expression and production of the engineered polypeptide.

Depending on the expression vector, manipulation of the isolated polynucleotide prior to insertion of the isolated polynucleotide into the vector may be desirable or necessary. Techniques for modifying polynucleotides and nucleic acid sequences using recombinant DNA methods are well known in the art. Guidance is provided below: Sambrook et al, 2001, MolecuLar Cloning: A Laboratory Manual, Third Edition, Cold Spring Harbor Laboratory Press; and Current Protocols in MolecuLar Biology, edited by Ausubel. F., GreenePub. Associates, 1998, updated 2010.

In another aspect, the present disclosure also relates to recombinant expression vectors, depending on the type of host they are to be introduced into, including a polynucleotide encoding an engineered ketoreductase polypeptide or variant thereof, and one or more expression regulatory regions, such as promoters and terminators, origin of replication and the like. Alternatively, the nucleic acid sequences of the present disclosure can be expressed by inserting the nucleic acid sequence or the nucleic acid construct comprising the sequence into an appropriate expression vector. In generating an expression vector, the coding sequence is located in the vector such that the coding sequence is linked to a suitable control sequence for expression.

The recombinant expression vector may be any vector (e.g., plasmid or virus) that can be conveniently used in the recombinant DNA procedures and can result in the expression of a polynucleotide sequence. The choice of vector will generally depend on the compatibility of the vector with the host cells to be introduced into. The vector may be a linear or closed circular plasmid. The expression vector may be an autonomously replicating vector, i.e., a vector that exists as an extrachromosomal entity whose replication is independent of chromosomal replication, such as a plasmid, extrachromosomal element, minichromosome, or artificial chromosomes. The vector may contain any tools for ensuring self-replication. Alternatively, the vector may be a vector that, when introduced into a host cell, integrates into the genome and replicates with the chromosome into which it is integrated. Moreover, a single vector or plasmid or two or more vectors or plasmids that together comprise the total DNA to be introduced into the genome of the host cell may be used.

Many expression vectors useful to the embodiments of the present disclosure are commercially available. Exemplary expression vectors can be prepared by inserting a polynucleotide encoding an improved ketoreductase polypeptide to the plasmid pACYC-Duet-1 (Novagen).

In another aspect, the present disclosure provides host cells comprising polynucleotides encoding engineered ketoreductase polypeptides of the present disclosure, the polynucleotide is linked to one or more control sequences for expression of ketoreductase in the host cell. Host cells for expression of polypeptides encoded by expression vectors of the present disclosure are well known in the art, including, but are not limited to, bacterial cells such as Escherichia coli, Arthrobacter spp. KNK168, Streptomyces, and Salmonella typhimurium cells; fungal cells such as yeast cells (e.g., Saccharomyces cerevisiae or Pichia pastoris); insect cells such as Drosophila S2 and Spodoptera Sf9 cells; animal cells such as CHO, COS, BHK, 293 and Bowes melanoma cells; and plant cells. An exemplary host cell is E. coli BL21 (DE3). The above host cells may be wild-type or engineered cells through genomic edition, such as knockout of the wild-type ketoreductase gene carried in the host cell's genome. Suitable media and growth conditions for the above host cells are well known in the art.

Polynucleotides used to express engineered ketoreductase can be introduced into the cells by a variety of methods known in the art. Techniques comprise, among others, electroporation, bio-particle bombardment, liposome-mediated transfection, calcium chloride transfection, and protoplast fusion. Different methods of introducing polynucleotides into cells are obvious to those skilled in the art.

### 2.4 Process of Producing an Engineered Ketoreductase Polypeptides

When the sequence of the engineered polypeptide is known, the encoding polynucleotide may be prepared by standard solid-phase methods according to known synthetic methods. In some embodiments, fragments of up to about 100 bases can be synthesized separately and then ligated (e.g., by enzymatic or chemical ligation methods or polymerase-mediated methods) to form any desired contiguous sequence. For example, the polynucleotides and oligonucleotides of the present disclosure can be prepared by chemical synthesis using, for example, the classical phosphoramidite method described by Beaucage et al, 1981, TetLett 22:1859-69, or the method described by Matthes et al, 1984, EMBOJ. 3:801-05, as typically practiced in automated synthetic methods. According to the phosphoramidite method, oligonucleotides are synthesized, purified, annealed, ligated, and cloned into a suitable vector, for example, in an automated DNA synthesizer. In addition, essentially any nucleic acid is available from any of a variety of commercial sources.

In some embodiments, the present disclosure also provides a process for preparing or producing an engineered ketoreductase polypeptide, wherein the process comprises culturing a host cell capable of expressing a polynucleotide encoding the engineered polypeptide under culture conditions suitable for expression of the polypeptide. In some embodiments, the process of preparing a polypeptide further comprises isolating the polypeptide. The engineered polypeptide may be expressed in a suitable cell and isolated (or recovered) from the host cell and/or culture medium using any one or more of the techniques known for protein purification, the techniques for protein purification include, among others, lysozyme treatment, sonication, filtration, salting out, ultracentrifugation and chromatography.

### 2.5 Methods Using Engineered Ketoreductases and Compounds Prepared Therewith

In another aspect, the improved engineered ketoreductase polypeptides described herein convert carbonyl compounds to chiral alcohols in the presence of the cofactor NADH. The present disclosure also provides processes of preparing a wide range of compounds (I) or structural analogs thereof using an engineered ketoreductase polypeptides disclosed herein. In some embodiments, engineered ketoreductase polypeptides can be used in a process of preparing compounds of structural formula (I):

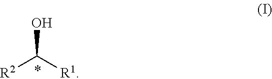

The alcohol product of formula (I) has the indicated stereochemical configuration at the chiral center marked with an *; the alcohol product of structure (I) is in enantiomeric excess over the other isomer, wherein

R¹ is optionally substituted or unsubstituted aryl or heteroaryl, or optionally substituted or unsubstituted C₁-C₈ hydrocarbon group, or may be a cyclic hydrocarbon group or a heterocyclic group.

R² is an optionally substituted or unsubstituted C₁-C₆ hydrocarbon group, a halogen (such as —F, —Cl, —Br and —I), an alkenyl, an alkynyl, an aryl, a heteroaryl, —NO₂, —NO, —SO₂R′ or —SOR′, —SR′, —NR′R′, —OR′, —CO₂R′ or —COR′, —C(O)NR′, —SO₂NH₂ or —SONH₂, —CN, —CF₃; wherein each R′ is independently selected from —H, C₁-C₄ hydrocarbon group, halogen, C₁-C₈ hydrocarbon group, C₂-C₁₂ alkenyl group, C₂-C₁₂ alkynyl group, cyclic hydrocarbon group, aryl group or heterocyclic group.

The process comprises contacting a carbonyl substrate of structural formula (II)

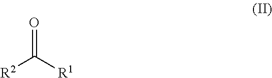

with a ketoreductase polypeptide under reaction conditions suitable for converting the carbonyl substrate to an alcohol product, wherein said ketoreductase polypeptide is an engineered ketoreductase polypeptide as described herein.

In some embodiments, the engineered ketoreductase polypeptides of the present disclosure are capable of asymmetrically reducing ethyl 4-chloroacetoacetate to ethyl (R)-4-chloro-3-hydroxybutyrate with higher activity than SEQ ID NO: 2. The engineered ketoreductase polypeptide provided by the present invention has better activity, stability, as well as solvent and substrate tolerance than the wild-type ketoreductase of SEQ ID NO: 2, and are able to catalyze the asymmetric reduction of ethyl 4-chloroacetoacetate to ethyl (R)-4-chloro-3-hydroxybutyrate more efficiently; or at high substrate loadings, the engineered ketoreductase polypeptide provided by the present invention are still capable of catalyzing the asymmetric reduction of ethyl 4-chloroacetoacetate to ethyl (R)-4-chloro-3-hydroxybutyrate without inhibition. These engineered ketoreductase polypeptides may comprise an amino acid sequence that differs from the sequence of SEQ ID NO: 2 in one or more residue positions selected from: X18, X22, X42, X46, X47, X48, X53, X67, X79, X80, X94, X96, X111, X115, X149, X204, X211 X229, X238, X239, X262, X282, X283, X284, X286, X303, X327. In some embodiments, the engineered ketoreductase polypeptide comprises an amino acid sequence having at least one of the following features: V18I, A22K, I42L, D46G, D46F, D46V, M47I, M47S, P48R, P48H I53Q, I53G, I53P, I53T, I53S, I53H, I53N, I53R, I53K, T67V, T67I, E79K, T80V, A94R, A94Y, A94C, A94H, H96R, E111H, T115R, T115C, T115S, T115H, V149A L204I, L211F, D229A, E238R, E238K, G239S, I262V, F282Q, F282G, M283N, M283D, I284M, I284L, I284V, F286V, F286H, F286R, D303E, T327L, T327E; or in addition to the abovementioned differences, the engineered ketoreductase polypeptides comprise insertions or deletions of one or more amino acid residues.

More specifically, in some embodiments, the engineered ketoreductase polypeptide improved upon SEQ ID NO: 2 comprises an amino acid sequence selected from SEQ ID NO: 4, 6, 8, 10, 12, 14, 16, 18, 20, 22, 24, 26, 28, 30, 32, 34, 36, 38, 40, 42, 44, 46, 48, 50, 52, 54, 56 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128.

In some embodiments, the engineered ketoreductase polypeptides comprise amino acid sequences that have at least 80%, 85%, 86%, 87%, 88 89%, 90%, 91%, 92%, 93%, 94%, 95%, 96%, 97%, 98%, 99% or more sequence identity to the reference amino acid sequence selected from SEQ ID No: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 72, 74, 76, 78, 80, 82, 84, 86, 88, 90, 92, 94, 96, 98, 100, 102, 104, 106, 108, 110, 112, 114, 116, 118, 120, 122, 124, 126, 128.

In some embodiments of the process, the chiral alcohol product of structural formula (I) is ethyl (R)-(+)-4-chloro-3-hydroxybutyrate:

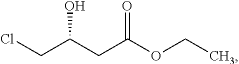

and the carbonyl substrate of structural formula (II) is ethyl 4-chloroacetoacetate:

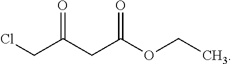

In some embodiments of the process, the chiral alcohol product of structural formula (I) is methyl (S)-(+)-3-hydroxybutyrate:

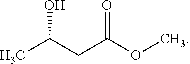

and the carbonyl substrate of structural formula (II) is methyl acetoacetate:

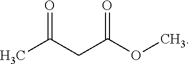

In some embodiments of the process, the chiral alcohol product of structural formula (I) is ethyl (S)-(+)-3-hydroxybutyrate:

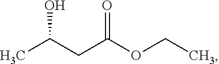

and the carbonyl substrate of structural formula (II) is ethyl acetoacetate:

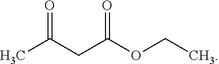

In some embodiments of the process, the chiral alcohol product of structural formula (I) is (S)-1-[3,5-bis(trifluoromethyl)phenyl]ethanol:

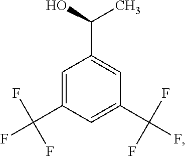 [SMILES: C[C@H](O)c1cc(C(F)(F)F)cc(C(F)(F)F)c1]

and the carbonyl substrate of structural formula (II) is 3,5-bis(trifluoromethyl)acetophenone:

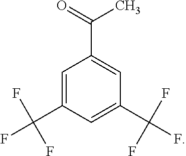 [SMILES: CC(=O)c1cc(C(F)(F)F)cc(C(F)(F)F)c1]

In some embodiments of the process, the chiral alcohol product of structural formula (I) is (S)-1,3-butanediol:

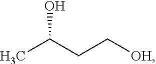

and the carbonyl substrate of structural formula (II) is 4-hydroxy-2-butanone:

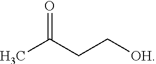

In some embodiments of the process, the chiral alcohol product of structural formula (I) is (2S,5S)-2,5-hexanediol:

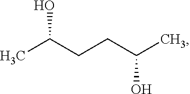

and the carbonyl substrate of structural formula (II) is 2,5-hexanedione:

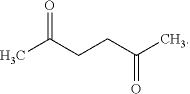

In some embodiments of the process, the chiral alcohol product of structural formula (I) is (S)-1-tert-butoxycarbonyl-3-hydroxypiperidine:

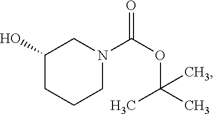

and the carbonyl substrate of structural formula (II) is N-tert-butoxycarbonyl-3-piperidone:

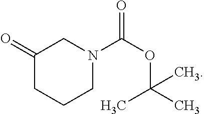

In some embodiments of the process, the chiral alcohol product of structural formula (I) is (S)-(−)-3-chloro-1-phenyl-1-propanol:

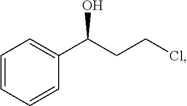 [SMILES: O[C@@H](CCCl)c1ccccc1]

and the carbonyl substrate of structural formula (II) is 3-chloropropiophenone:

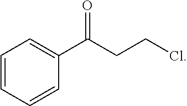 [SMILES: O=C(CCCl)c1ccccc1]

In some embodiments of the process, the chiral alcohol product of structural formula (I) is methyl (R)-(+)-4-chloro-3-hydroxybutyrate:

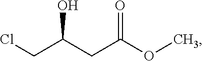

and the carbonyl substrate of structural formula (II) is methyl 4-chloroacetoacetate:

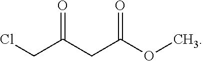

In some embodiments of the process, the chiral alcohol product of structural formula (I) is (2S,3S)-2,3-butanediol:

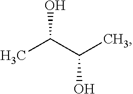

and the carbonyl substrate of structural formula (II) is 2,3-butanedione:

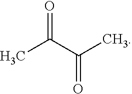

In some embodiments of the process, the chiral alcohol product of structural formula (I) is (2S,4S)-2,4-pentanediol:

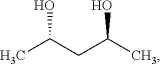

and the carbonyl substrate of structural formula (II) is acetylacetone:

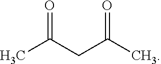

In some embodiments of the process, the chiral alcohol product of structural formula (I) is (2S,5S)-2,5-hexanediol:

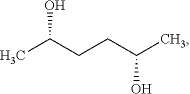

and the carbonyl substrate of structural formula (II) is 2,5-hexanedione:

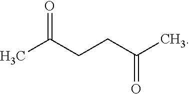

In some embodiments of the process, the chiral alcohol product of structural formula (I) is (3S,5S)-3,5-heptanediol:

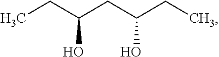

and the carbonyl substrate of structural formula (II) is 3,5-heptanedione:

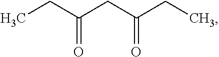

In some embodiments of the process, the chiral alcohol product of structural formula (I) is (3S,6S)-3,6-octanediol:

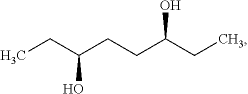

and the carbonyl substrate of structural formula (II) is 3,6-octanedione:

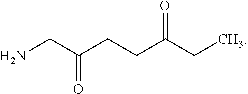

In some embodiments of the process, the chiral alcohol product of structural formula (I) is (3S,6S)-2,7-dimethyl-3,6-octanediol:

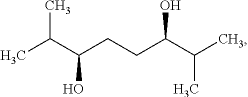

and the carbonyl substrate of structural formula (II) is 2,7-dimethyloctane-3,6-dione:

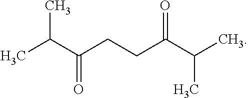

### BRIEF DESCRIPTION OF THE SEVERAL VIEWS OF THE DRAWING(S)

FIG. 1. Ketoreductase-catalyzed reduction of carbonyl compounds to alcohols

FIG. 2. Ketoreductase-catalyzed conversion of ethyl 4-chloroacetoacetate to ethyl (R)-(+)-4-chloro-3-hydroxybutyrate and the simultaneous conversion of isopropanol to acetone for the regeneration of NADH

FIG. 3. SDA-PAGE image of the enzyme solution after heat treatment

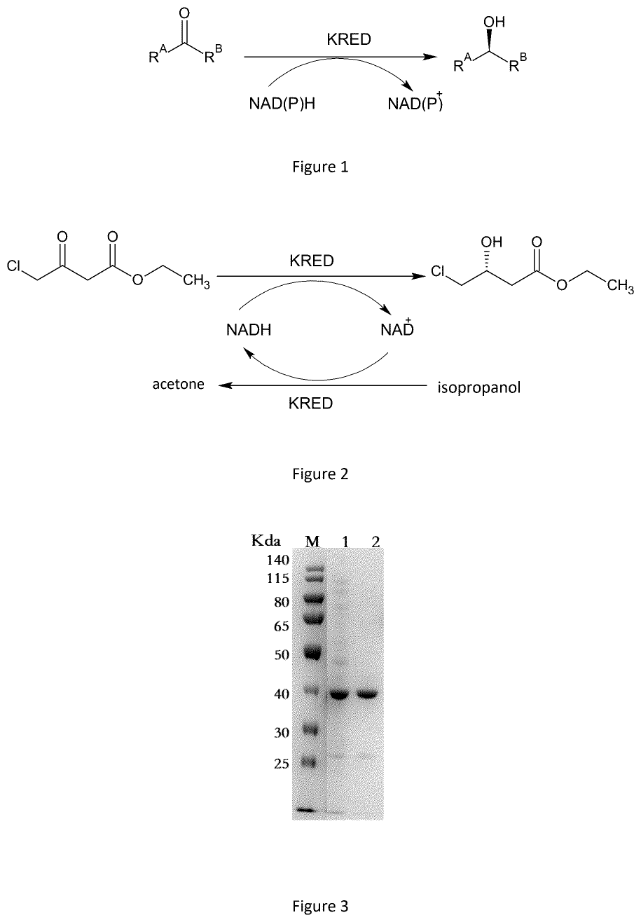

### DESCRIPTION OF SPECIFIC EMBODIMENTS OF THE INVENTION

The following examples further illustrate the present invention, but the present invention is not limited thereto. In the following examples, experimental methods with conditions not specified, were conducted at the commonly used conditions or according to the suppliers' suggestion.

### Example 1: Gene Cloning and Construction of Expression Vectors

The amino acid sequence of the wild-type ketoreductase from Rhodococcus erythropolis is shown in SEQ ID NO: 2 which can be retrieved from NCBI, and the corresponding nucleic acid was then synthesized by a vendor using conventional techniques in the art and cloned into the expression vector pACYC-Duet-1. The recombinant expression plasmid was transformed into E. coli BL21 (DE3) competent cells under the conditions of 42° C. and thermal shock for 90 seconds. The transformation was placed on LB agar plates containing chloramphenicol, which was then incubated overnight at 37° C. Recombinant transformants were obtained.

### Example 2: Expression of Ketoreductase Polypeptide, and Preparation of Wet Cells or Enzyme Solution Containing Ketoreductase Polypeptide

The enzyme solution of the present invention was prepared as follows: the recombinant E. coli BL21 (DE3) obtained in Example 1 was inoculated into 50 mL of LB medium containing chloramphenicol (peptone 10 g/L, yeast extract powder 5 g/L, sodium chloride 10 g/L, pH 7.0±0.2, 25° C.) in a 250 mL Erlenmeyer flask, which was then cultured in a shaking incubator at 30° C., 250 rpm overnight. When the OD₆₀₀ of the overnight culture reached 2, it was subcultured at the inoculum of 5% (v/v) into a 1.0 L flask containing 250 mL of TB medium (tryptone 12 g/L, yeast extract 24 g/L, disodium hydrogen phosphate 9.4 g/L, dipotassium hydrogen phosphate 2.2 g/L, lactose 6 g/L, pH 7.0±0.2, 30° C.). The medium was also supplemented with ZnCl₂ at a final concentration of 1 mM and incubated at 30° C. with shaking at 250 rpm, during which lactose induced the expression of ketoreductase polypeptide. After about 20 h, the expression culture was centrifuged (8000 rpm, 10 min); after centrifugation, the supernatant was discarded and the cells were collected to obtain wet cells containing ketoreductase polypeptide. The wet cells can be used directly in the reaction or stored frozen at −20° C. until use.

The wet cells can also be used directly in the preparation of the enzyme solution. The wet cells were suspended in 30 mL of phosphate buffer (PB, pH 7.0), sonicated in an ice bath and the supernatant was collected by centrifugation (4000 rpm, 15 min) to obtain the enzyme solution containing the ketoreductase polypeptide.

According to the shake flask preparation procedure, it can be proportionally scaled-down to 96-well plate for culture to obtain the culture solution, and the culture medium is removed by centrifugation to obtain the wet cells; the enzyme solution can be obtained by the chemical lysis method known in the art.

### Example 3: Heat Treatment

25 mL of the enzyme solution containing ketoreductase polypeptide corresponding to SEQ ID No: 38 was prepared by the method described in Example 2. It was loaded into a 50 mL centrifuge tube and placed in a water bath at 68° C., stirred to uniformly incubate the enzyme solution at 68° C. After 4 hours, the enzyme solution was centrifuged (4000 rpm, 30 min) and the supernatant was collected to obtain the heat-treated enzyme solution. The heat-treated enzyme solution sample was subjected to SDS-PAGE analysis, and the electrophoresis result was shown in FIG. 3. Sample 1 is the enzyme solution of SEQ ID No: 38 without heat treatment, and sample 2 is the enzyme solution of SEQ ID No: 38 after heat treatment. The results of FIG. 3 show that a large amount of background protein can be removed by heat treatment at [68° C., 4 h], and the enzyme of SEQ ID No: 38 has good thermal stability.

### Example 4: Construction of a Ketoreductase Mutant Library

All the reagents used here are commercial reagents, and preferably the Quikchange kit (supplier: Agilent) was used. The sequence design of the mutagenesis primers was performed according to the instructions of the kit. The construction of a site-saturation mutagenesis library is as following. The PCR reaction consisted of 10 μl of 5× Buffer, 1 μl of 10 mM dNTP, 1 μl of plasmid DNA template (50 ng/μl), 0.75 μl (10 uM) each of the upstream and downstream primers, 0.5 μl of high fidelity enzyme and 36 μl of ddH2O, The PCR primer has a NNK codon at the mutation position.

PCR amplification steps: (1) 98° C. pre-denaturation 3 min; (2) 98° C. denaturation 10 s; (3) annealing and extension 3 min at 72° C.; steps of (2)˜(3) repeated 25 times; (5) extension 10 min at 72° C.; (6) cooling to 4° C., 2 μl of DpnI was added to the PCR product and the plasmid template was eliminated by overnight digestion at 37° C. The digested PCR product was transformed into E. coli BL21 (DE3) competent cells and plated on LB agar plates containing chloramphenicol to obtain a mutagenesis library.

### Example 5: High-Throughput Screening of Ketoreductase Mutant Library

For expression of the ketoreductase mutant library, wet cells were obtained by culturing and inducing expression using 96-well plates as described in Example 2.

200 μL/well PB buffer (0.1 M phosphate buffer, pH 7.0) was added the deep-well plate containing wet cells, sealed and placed on a plate shaker at 700 rpm for 1 h. Afterwards, 40 μL of the solution per well was taken into a 96-well plate pre-filled with reaction stock solution (160 μL/well). The 96-well plates were then heat-sealed with aluminum film and placed on a shaker at 30° C. and 200 rpm to start the reaction. After 24 h of reaction, the reaction was quenched by adding 1 mL of ethyl acetate in each well, then plate placed on a plate shaker for 30 min (800 rpm), and then centrifuged (4000 rpm, 10 min), and the supernatant after centrifugation was taken for GC analysis to detect the conversion.

The reaction stock solution was prepared as follows: a) dissolve ethyl 4-chloroacetoacetate in isopropanol, b) dissolve NAD+ in PB buffer, and mix a) and b) to obtain the reaction stock solution with the final concentration of [ethyl 4-chloro-acetoacetate 304.5 g/L, isopropanol 31.25% (v/v), NAD+0.625 g/L, PB 0.1M, pH 7.0].

### Example 6: Analytical methods of Ethyl (R)-(+)-4-chloro-3-hydroxybutyrate

### GC Analysis Method:

The chromatographic column was Agilent DB-WAX 15 m*0.25 mm*0.25 μm, the carrier gas was N2, the detector was FID, the inlet temperature was 140° C., the splitting ratio was 10:1, the detector temperature was 300° C., the injection volume was 2 μL, the column temperature was 100° C., and the temperature was ramped up to 140° C. at 15° C./min and maintained for 3.9 min, where the retention time of ethyl 4-chloro-acetoacetate was 3.8 min and that of ethyl (R)-(+)-4-chloro-3-hydroxybutyrate was 5.4 min.

### HPLC Chiral Analysis Method:

The chromatographic column was Daicel CHIRALCEL AD-H 250 mm*4.6 mm*5 μm, mobile phase: 95% hexane+5% isopropanol, column temperature: 30° C., injection volume: 20 μL, flow rate: 1 ml/min, wavelength: 210 nm, where the retention time of ethyl (R)-(+)-4-chloro-3-hydroxybutyrate was 11.5 min, the retention time of ethyl (S)-(−)-4-chloro-3-hydroxybutyrate was 10.4 min, and ethyl 4-chloroacetoacetate had a retention time of 4.1 min.

### Example 7: Fermentation Expression

A single microbial colony of E. coli BL21 (DE3) containing a plasmid bearing the gene of target engineered ketoreductase was inoculated into 50 mL LB broth containing 30 g/mL chloramphenicol (5.0 g/L Yeast Extract, 10 g/L Tryptone, 10 g/L NaCl). Cells were incubated for about 16 hours with shaking at 250 rpm in a 30° C. shaker. When the OD₆₀₀ of the culture reached 3.5 to 5.0, the culture was used to inoculate a fermentor.

A 1.0 L fermentor containing 0.6 L of base medium was sterilized at 121° C. for 30 min. The fermentor was charged with ZnCl₂ at a final concentration of 1.0 mM, mixed well and then inoculated with the abovementioned culture. Temperature of fermentor was maintained at 37° C. The medium in fermentor was agitated at 200-1000 rpm and air was supplied to the fermentation vessel at 0.4-0.8 L/min to maintain the dissolved oxygen level at 35% saturation or greater. The culture was maintained at pH 7.0 by addition of 25-28% v/v ammonium hydroxide. Cell growth was maintained by feeding a feed solution containing 500 g/L of dextrose glucose monohydrate, 12 g/L of ammonium chloride, and 5 g/L of magnesium sulfate heptahydrate. The culture was incubated up to 8 h. After the OD₆₀₀ of culture reached 35±5, the temperature of fermentor was decreased and maintained at 30° C., and the expression of ketoreductase was induced by the addition of monohydrated-α-lactose to a final concentration of 15 g/L. Fermentation process then continued for additional 16 hours. After the fermentation process was complete, cells were harvested using a Thermo Multifuge X3R centrifuge at 8000 rpm for 10 min at 4° C. Harvested cells were used directly in the downstream process or stored frozen at −20° C.

### Example 8: Reaction Process for the Synthesis of Ethyl (R)-(+)-4-chloro-3-hydroxybutyrate Catalyzed by Engineered Ketoreductase

A representative reaction process at 100 mL reaction volume and the workup process are shown below. To a 250 mL reaction flask, 1.0 g of SEQ ID NO: 34 wet cells, 15 mL of 0.1 M pH7.6 phosphate buffer, 10 mg of NAD+, 25 mL of isopropanol and 40 mL of toluene were added, and finally 24.4 g of ethyl 4-chloroacetoacetate was added to the reaction vessel and stirred. The temperature of reaction was maintained at 30° C. and the stirring speed was 250 rpm. The reaction was carried out up to 24 h, during which the pH was controlled in real time and a vacuum of 0.07 MPa was applied once to remove the acetone produced by the reaction at 16 h. The conversion at end point was 99.5%.

After the reaction, the reaction solution was stratified by standing, with the lower layer being aqueous phase and the upper layer being the toluene layer. The toluene layer was subjected to distillation under reduced pressure to remove isopropanol, acetone and toluene, and then 22.4 g of the product ethyl (R)-(+)-4-chloro-3-hydroxybutyrate was collected by raising the temperature of distillation. The product yield was 90.7%, the purity was 99%, and ee≥99.8%.

### Example 9: Reaction Process for the Synthesis of Ethyl (R)-(+)-4-chloro-3-hydroxybutyrate Catalyzed by Engineered Ketoreductase

A representative reaction process at 100 mL reaction volume and the workup process are shown below. To a 250 mL reaction flask, 1.0 g of SEQ ID NO: 6 wet cells, 15 mL of 0.1 M pH7.6 phosphate buffer, 10 mg of NAD+, 25 mL of isopropanol and 35 mL of toluene were added, and finally 30.5 g of ethyl 4-chloroacetoacetate was added to the reaction vessel and stirred. The temperature of reaction was maintained at 30° C. and the stirring speed was 250 rpm. The reaction was carried out up to 24 h, during which the pH was controlled in real time and a vacuum of 0.07 MPa was applied once to remove the acetone produced by the reaction at 16 h. The conversion at end point was 99.5%.

After the reaction, the reaction solution was stratified by standing, with the lower layer being aqueous phase and the upper layer being the toluene layer. The toluene layer was subjected to distillation under reduced pressure to remove isopropanol, acetone and toluene, and 28.1 g of the product ethyl (R)-(+)-4-chloro-3-hydroxybutyrate was collected by raising the temperature of distillation. The product yield was 91%, the purity was 99%, and ee≥99.8%.

### Example 10: Reaction Process for the Synthesis of Ethyl (R)-(+)-4-chloro-3-hydroxybutyrate Catalyzed by Engineered Ketoreductase

A representative reaction process at 100 mL reaction volume and the workup process are shown below. To a 250 mL reaction flask, 1.0 g of SEQ ID NO: 56 wet cells, 15 mL of 0.1 M pH7.6 phosphate buffer, 10 mg of NAD+, 25 mL of isopropanol and 45 mL of toluene were added, and finally 24.4 g of ethyl 4-chloroacetoacetate was added to the reaction vessel and stirred. The temperature of reaction was maintained at 30° C. and the stirring speed was 250 rpm. The reaction was carried out up to 24 h, during which the pH was controlled in real time and a vacuum of 0.07 MPa was applied once to remove the acetone produced by the reaction at 16 h. The conversion at end point was 99.5%.

After the reaction, the reaction solution was stratified by standing, with the lower layer being aqueous phase and the upper layer being the toluene layer. The toluene layer was subjected to distillation under reduced pressure to remove isopropanol, acetone and toluene, and 22.7 g of the product ethyl (R)-(+)-4-chloro-3-hydroxybutyrate was collected raising the temperature of distillation. The product yield was 91.9%, the purity was 99%, and ee≥99.8%.

### Example 11: Reaction Process for the Synthesis of Ethyl (R)-(+)-4-chloro-3-hydroxybutyrate Catalyzed by Engineered Ketoreductase

A representative reaction process at 100 mL reaction volume and the workup process are shown below. To a 250 mL reaction flask, 1.0 g of SEQ ID NO: 70 wet cells, 15 mL of 0.1 M pH7.6 phosphate buffer, 10 mg of NAD+, 25 mL of isopropanol and 27 mL of toluene were added, and finally 40 g of ethyl 4-chloroacetoacetate was added to the reaction vessel and stirred. The temperature of reaction was maintained at 30° C. and the stirring speed was 250 rpm. The reaction was carried out up to 24 h, during which the pH was controlled in real time and a vacuum of 0.07 MPa was applied once to remove the acetone produced by the reaction at 16 h. The conversion at end point was 99.5%.

After the reaction, the reaction solution was stratified by standing, with the lower layer being aqueous phase and the upper layer being the toluene layer. The toluene layer was subjected to distillation under reduced pressure to remove isopropanol, acetone and toluene, and 37 g of the product ethyl (R)-(+)-4-chloro-3-hydroxybutyrate was collected raising the temperature of distillation. The product yield was 91.4%, the purity was 99%, and ee≥99.8%.

### Example 12: Reaction Process for the Synthesis of Ethyl (R)-(+)-4-chloro-3-hydroxybutyrate Catalyzed by Engineered Ketoreductase

A representative reaction process at 100 mL reaction volume and the workup process are shown below. To a 250 mL reaction flask, 1.0 g of SEQ ID NO: 92 wet cells, 15 mL of 0.1 M pH7.6 phosphate buffer, 10 mg of NAD+, 25 mL of isopropanol and 35 mL of toluene were added, and finally 30.5 g of ethyl 4-chloroacetoacetate was added to the reaction vessel and stirred. The temperature of reaction was maintained at 30° C. and the stirring speed was 250 rpm. The reaction was carried out up to 24 h, during which the pH was controlled at 6.5-7.5 in real time and a vacuum of 0.07 MPa applied once to remove the acetone produced by the reaction at 16 h. The conversion at end point was 99.5%.

After the reaction, the reaction solution was stratified by standing, with the lower layer being aqueous phase and the upper layer being the toluene layer. The toluene layer was subjected to distillation under reduced pressure to remove isopropanol, acetone and toluene, and 28.1 g of the product ethyl (R)-(+)-4-chloro-3-hydroxybutyrate was collected by raising the temperature of distillation. The product yield was 91%, the purity was 99%, and ee≥99.8%.

### Example 13: Reaction Process the for Synthesis of Ethyl (R)-(+)-4-chloro-3-hydroxybutyrate Catalyzed by Engineered Ketoreductase

A representative reaction process at 100 mL reaction volume and the workup process are shown below. To a 250 mL reaction flask, 1.0 g of SEQ ID NO: 92 wet cells, 15 mL of 0.1 M pH7.6 phosphate buffer, 10 mg of NAD+, 25 mL of isopropanol and 35 mL of toluene were added, and finally 30.6 g of ethyl 4-chloroacetoacetate was added to the reaction vessel and stirred. The temperature of reaction was maintained at 30° C. and the stirring speed was 250 rpm. The reaction was carried out up to 24 h, during which the pH was controlled at 5.5-6.5, and a vacuum of 0.07 MPa was applied once to remove the acetone produced by the reaction at 16 h. The conversion at end point was 99.5%.

After the reaction, the reaction solution was stratified by standing, with the lower layer being aqueous phase and the upper layer being the toluene layer. The toluene layer was subjected to distillation under reduced pressure to remove isopropanol, acetone and toluene, and 28.5 g of the product ethyl (R)-(+)-4-chloro-3-hydroxybutyrate was collected by raising the temperature of distillation. The product yield was 92%, the purity was 99%, and ee≥99.8%.

### Example 14: Reaction Process for the Synthesis of Ethyl (R)-(+)-4-chloro-3-hydroxybutyrate Catalyzed by Engineered Ketoreductase

The following is the reaction process at a 100 mL volume in pure aqueous phase. To a 250 mL reaction flask, 2.0 g of SEQ ID NO: 56 wet cells, 55 mL of 0.1 M pH7.6 phosphate buffer, 50 mg of NAD+, 25 mL of isopropanol, and finally 24.4 g of ethyl 4-chloroacetoacetate were added and stirred. The temperature of reaction was maintained at 30° C. and the stirring speed was 250 rpm. The reaction was carried out up to 24 h, during which the pH was controlled. The pH would drop rapidly during the reaction, and it was difficult to stabilize the pH by adding base; as the base was added, the color of reaction solution changed from light red until it turned black. The conversion was 87.5% at end point and the yield of ethyl (R)-(+)-4-chloro-3-hydroxybutyrate was 75%.

### Example 15: Comparison of the Catalytic Performance of Engineered Ketoreductase SEQ ID No: 102 with Wild-Type Ketoreductase SEQ ID No: 2 in Catalyzing the Reaction to Produce Ethyl (R)-(+)-4-chloro-3-hydroxybutyrate

Wet cells of a) SEQ ID No: 2 and b) SEQ ID No: 102 were prepared according to the method described in Example 2. Wet cells of a) and b) were resuspended using 0.1 M PB at pH 7.0 to obtain cell suspension of c) 100 g/L of SEQ ID No: 2 and cell suspension d) 100 g/L of SEQ ID No: 102, respectively. Part of cell suspension c) and cell suspension d) were taken out for heat treatment at 68° C. for 4 h to obtain e) and f), respectively.

Four 30 mL reaction flasks were taken, and 1.218 g of ethyl 4-chloroacetoacetate and 1.25 mL of isopropanol were added to each of the reaction flasks. Then 4 different cell suspensions were added to each of the 4 reaction flasks: 0.5 mL of c), 0.5 mL of d), 0.5 mL of e), 0.5 mL of f), followed by 0.25 mL of stock solution (2 g/L) of NAD+ and 2.25 mL of toluene. The final substrate loading in the reaction flask was 240 g/L, the final concentration of isopropanol was 25% v/v, the final concentration of NAD+ was 0.5 g/L, and the final concentration of toluene was 45% v/v. The reactions were carried out with controlled temperature of 30° C. and a stirring speed of 400 rpm with magnetic stirrer. 50 μL of the reaction solution was sampled after 24 h, and it was quenched by adding 950 μL of ethyl acetate for analysis as described in Example 6. The results are shown in Table 2.

TABLE 2

| Enzymes | Conversion |
|---|---|
| c) non-heat treated SEQ ID No: 2 | 8.61% |
| d) non-heat treated SEQ ID No: 102 | >99.9% |
| e) [68° C., 4 h] heat-treated SEQ ID No: 2 | 4.18% |
| f) [68° C., 4 h] heat-treated SEQ ID No: 102 | >99.9% |

### Example 16: Reaction Process for the Synthesis of Ethyl (R)-(+)-4-chloro-3-hydroxybutyrate Catalyzed by Engineered Ketoreductase

A representative reaction process at 100 mL reaction volume and the workup process are shown below. To a 250 mL reaction flask, 1.0 g of SEQ ID NO: 118 wet cells, 10 mL of 0.1 M pH7.6 phosphate buffer, 10 mg of NAD+, 25 mL of isopropanol and 45 mL of toluene were added, and finally 24.6 g of ethyl 4-chloroacetoacetate was added to the reaction vessel and stirred. The temperature of reaction was maintained at 40° C. and the stirring speed was 250 rpm. The reaction was carried out up to 24 h, during which the pH was controlled at 6.5-7.5, and a vacuum of 0.07 MPa was applied to remove the acetone produced by the reaction at 16 h. The conversion at end point was 99.6%.

After the reaction, the reaction solution was stratified by standing, with the lower layer being aqueous phase and the upper layer being the toluene layer. The toluene layer was subjected to distillation under reduced pressure to remove isopropanol, acetone and toluene, and 22.8 g of the product ethyl (R)-(+)-4-chloro-3-hydroxybutyrate was collected by raising the temperature of distillation. The product yield was 91.5%, the purity was 99%, and ee≥99.8%.

### Example 17: Reaction Process for the Synthesis of Ethyl (R)-(+)-4-chloro-3-hydroxybutyrate Catalyzed by Engineered Ketoreductase

A representative reaction process at 100 mL reaction volume and the workup process are shown below. To a 250 mL reaction flask, 1.0 g of SEQ ID NO: 120 wet cells, 15 mL of 0.1 M pH7.6 phosphate buffer, 10 mg of NAD+, 25 mL of isopropanol and 35 mL of toluene were added, and finally 30.6 g of ethyl 4-chloroacetoacetate was added to the reaction vessel and stirred. The temperature of reaction was maintained at 30° C. and the stirring speed was 250 rpm. The reaction was carried out up to 24 h, during which the pH was controlled at 6.5-7.5, and 5 mL of isopropanol was added when the reaction reached 16 h. The conversion at end point was 99.0%.

After the reaction, the reaction solution was stratified by standing, with the lower layer being aqueous phase and the upper layer being the toluene layer. The toluene layer was subjected to distillation under reduced pressure to remove isopropanol, acetone and toluene, and 28.2 g of the product ethyl (R)-(+)-4-chloro-3-hydroxybutyrate was collected by raising the temperature of distillation. The product yield was 91%, the purity was 98.5%, and ee≥99.8%.

### Example 18: Reaction Process for the Synthesis of Ethyl (R)-(+)-4-chloro-3-hydroxybutyrate Catalyzed by Engineered Ketoreductase

A representative reaction process at 100 mL reaction volume and the workup process are shown below. To a 250 mL reaction flask, 2.0 g of SEQ ID NO: 120 wet cells, 15 mL of 0.1 M pH7.6 phosphate buffer, 20 mg of NAD+, 30 mL of isopropanol and 20 mL of toluene were added, and finally 42.6 g of ethyl 4-chloroacetoacetate was added to the reaction vessel and stirred. The temperature of reaction was maintained at 30° C. and the stirring speed was 250 rpm. The reaction was carried out up to 24 h, during which the pH was controlled at 6.5-7.5, and a vacuum of 0.07 MPa was applied to remove the acetone produced by the reaction at 16 h. The conversion at end point was 96.0%.

After the reaction, the reaction solution was stratified by standing, with the lower layer being aqueous phase and the upper layer being the toluene layer. The toluene layer was subjected to distillation under reduced pressure to remove isopropanol, acetone and toluene, and 39.3 g of the product ethyl (R)-(+)-4-chloro-3-hydroxybutyrate was collected by raising the temperature of distillation. The product yield was 91.5%, the purity was 95%, and ee≥99.8%.

### Example 19: Synthesis of (S)-(+)-1,3-butanediol Catalyzed by Engineered Ketoreductase

To a 30 mL reaction flask, 0.25 g of SEQ ID No: 4 wet cells was added, then 0.25 g of 4-hydroxy-2-butanone, 0.5 mL of isopropanol and 0.25 mL of NAD+(10 g/L of stock solution) were added to the reaction flask. The final reaction volume in the reaction flask was topped up to 5.0 mL with 0.01 M PB at pH 7, and the final concentration of the substrate (4-hydroxy-2-butanone) in the reaction flask was 50 g/L, the final concentration of isopropanol was 10% v/v, and the final concentration of NAD+ was 0.5 g/L. The reaction flask was placed on an IKA magnetic stirrer at 30° C. with the stirring speed set to 400 rpm to start the reaction. After the reaction was carried out for 24 h, samples were taken from the reaction flask and processed and analyzed. The results are shown in Table 3.

TABLE 3

| | Wet cell | | ee of product (S)- |
|---|---|---|---|
| Enzyme | loading | Conversion | (+)-1,3-butanediol |
| SEQ ID No: 4 | 50 g/L | 70.16% | 96.41% |

### Analysis Method:

### Analysis Method for Conversion:

The chromatographic column was Agilent DB-WAX 15 m*0.25 mm*0.25 μm, the carrier gas was N2, the detector was FID, the inlet temperature was 250° C., the split ratio was 28:1, the detector temperature was 300° C., the injection volume was 1 μL, the column temperature was 130° C., and the temperature was ramped up to 150° C. at a rate of 10° C./min, and then to 160° C. at a rate of 20° C./min. The retention time of 4-hydroxy-2-butanone was 1.5 min, and the retention time of (S)-(+)-1,3-butanediol was 2.3 min.

### Chiral Analysis Method:

Sample pretreatment method: 200 μL of sample was added with 50 μL of MSTFA and 30 μL of anhydrous pyridine in a 1.5 mL centrifuge tube and mixed well, and the reaction was shaken for 30 min. The column was an Agilent CP-Chirasil Dex CB (CP7502) 25 m*0.25 mm*0.25 μm, the carrier gas was N2, and the detector was FID. The inlet temperature was 250° C., the splitting ratio was 28:1, the detector temperature was 300° C., the injection volume was 2 μL, the column temperature was 80° C., the stopping time was 35 min, and the retention time of (R)-(−)-1,3-butanediol was 20.7 min, and the retention time of (S)-(+)-1,3-butanediol was 23.0 min.

### Example 20: Synthesis of Methyl (S)-(+)-3-hydroxybutyrate Catalyzed by Engineered Ketoreductase

To a 30 mL reaction flask, 0.1 g of SEQ ID No: 18 wet cells was added, then 0.25 g of methyl acetoacetate, 0.5 mL of isopropanol and 0.25 mL of NAD+(10 g/L of stock solution) were added to the reaction flask. The final reaction volume in the reaction flask was topped up to 5.0 mL with 0.1 M PB at pH 7. The final concentration of substrate (methyl acetoacetate) was 50 g/L, the final concentration of isopropanol was 10% v/v and the final concentration of NAD+ was 0.5 g/L in the reaction flask. The reaction flask was placed on an IKA magnetic stirrer at 30° C. with the stirring speed set to 400 rpm to start the reaction. After the reaction was carried out for 24 h, samples were taken from the reaction flask and processed and analyzed. The results are shown in Table 4.

TABLE 4

| | | | ee of product |
|---|---|---|---|
| | Wet cell | | Methyl (S)-(+)-3- |
| Enzyme | loading | Conversion | hydroxybutyrate |
| SEQ ID No: 18 | 20 g/L | 99.9% | >99.9% |

### Analysis Method:

Analysis method for Conversion:

The chromatographic column was an Agilent HP-5 30 m*0.32 mm*0.25 μm, the carrier gas was N2, the detector was FID, the inlet temperature was 250° C., the split ratio was 50:1, the detector temperature was 300° C., the injection volume was 1 μL, the column temperature was 50° C., maintained for 5 min, then ramped up to 100° C. at a rate of 5° C./min, then ramped up to 225° C. at a rate of 50° C./min and kept for 5.5 min, in which the retention time of methyl acetoacetate was 9.5 min and that of methyl (S)-(+)-3-hydroxybutyrate was 8.9 min.

### Chiral Analysis Method:

The chromatographic column was Agilent CP-Chirasil Dex CB (CP7502) 25 m*0.25 mm*0.25 μm, the carrier gas was N2, the detector was FID, the inlet temperature was 250° C., the splitting ratio was 50:1, the detector temperature was 250° C., the injection volume was 1 μL, the column temperature was 100° C., the stopping time was 15 min, the retention time of Methyl (R)-3-hydroxybutyrate was 6.6 min, and the retention time of Methyl (S)-(+)-3-hydroxybutyrate was 7.0 min.

### Example 21: Synthesis of Ethyl (S)-(+)-3-hydroxybutyrate Catalyzed by Engineered Ketoreductase

To a 30 mL reaction flask, 0.1 g of SEQ ID No: 70 wet cells was added, then 0.25 g of ethyl acetoacetate, 0.5 mL of isopropanol and 0.25 mL of NAD+(10 g/L of stock solution) were added to the reaction flask. The final reaction volume in the reaction flask was topped up to 5.0 mL with 0.1 M PB at pH 7. The final concentration of substrate (ethyl acetoacetate) was 50 g/L, the final concentration of isopropanol was 10% v/v and the final concentration of NAD+ was 0.5 g/L in the reaction flask. The reaction flask was placed on an IKA magnetic stirrer at 30° C. with the stirring speed set to 400 rpm to start the reaction. After the reaction was carried out for 24 h, samples were taken from the reaction flask and processed and analyzed. The results are shown in Table 5.

TABLE 5

| | Wet cell | | ee of product (S)- |
|---|---|---|---|
| Enzyme | loading | Conversion | (+)-3-hydroxybutyrate |
| SEQ ID No: 70 | 20 g/L | 99.9% | >99.9% |

### Analysis Method:

### Analysis Method for Conversion:

The chromatographic column was Agilent DB-WAX 15 m*0.25 mm*0.25 μm, the carrier gas was N2, the detector was FID, the inlet temperature was 250° C., the split ratio was 50:1, the detector temperature was 300° C., the injection volume was 1 μL, the column temperature was 120° C., and the retention time was 4.5 min, in which the retention time of ethyl acetoacetate was 2.4 min and the retention time of ethyl 3-hydroxybutyrate was 2.8 min.

### Chiral Analysis Method:

The chromatographic column was Agilent CP-Chirasil Dex CB (CP7502) 25 m*0.25 mm*0.25 μm, the carrier gas was N2, the detector was FID, the inlet temperature was 250° C., the splitting ratio was 50:1, the detector temperature was 250° C., the injection volume was 1 μL, the column temperature was 110° C., the stopping time was 11 min, the retention time of Ethyl (R)-3-hydroxybutyrate was 9.4 min, and the retention time of Ethyl (S)-(+)-3-Hydroxybutyrate was 9.7 min.

### Example 22: Synthesis of (2S,5S)-2,5-hexanediol Catalyzed by Engineered Ketoreductase

To a 30 mL reaction flask, 0.1 g of SEQ ID No: 128 wet cells was added, then 0.25 g of 2,5-hexanedione, 0.5 mL of isopropanol and 0.25 mL of NAD+(10 g/L of stock solution) was added to the reaction flask. The final reaction volume in the reaction flask was topped up with 0.1 M PB at pH 7 to 5.0 mL, and the final concentration of the substrate (2,5-hexanedione) in the reaction flask was 50 g/L, the final concentration of isopropanol was 10% v/v, and the final concentration of NAD+ was 0.5 g/L. The reaction flask was placed on an IKA magnetic stirrer at 30° C. with the stirring speed set to 400 rpm to start the reaction. After the reaction was carried out for 24 h, samples were taken from the reaction flask and processed and analyzed. The results are shown in Table 6.

TABLE 6

| | Wet cell | | ee of product |
|---|---|---|---|
| Enzyme | loading | Conversion | (2S,5S)-2,5-hexanediol |
| SEQ ID No: 128 | 20 g/L | 81.74% | >99.9% |

### Analysis Method:

### Analysis Method for Conversion:

The chromatographic column was Agilent DB-WAX 30 m*0.25 mm*0.25 μm, the carrier gas was N2, the detector was FID, the inlet temperature was 250° C., the splitting ratio was 20:1, the detector temperature was 300° C., the injection volume was 3 μL, the column temperature was 100° C. and maintained for 3 min, and then it was ramped up to 150° C. at a rate of 5° C./min and maintained for 3 min, where the retention time of 2, 5-Hexanedione was 7.1 min and the retention time of 2,5-hexanediol was 14.1 min.

### Chiral Analysis Method:

200 μL of the reaction sample dissolved in trichloromethane was added with 200 μL of trifluoroacetic anhydride and sealed with a permeable membrane, followed by evaporation of the dry solvent in a constant temperature water bath at 60° C. for 20 min, and then 200 μL of trichloromethane was added into the sample. The chromatographic column was an Agilent CP-Chirasil Dex CB (CP7502) 25 m*0.25 mm*0.25 μm, the carrier gas was N2, the detector was FID, the inlet temperature was 270° C., the splitting ratio was 20:1, the detector temperature was 300° C., the injection volume was 1 μL, the column temperature was 70° C., the stopping time was 30 min, the retention time of (2S,5S)-2,5-hexanediol was 15.0 min and the retention time of (2R,5R)-2,5-hexanediol was 16.7 min.

### Example 23: Synthesis of (S)-1-[3,5-bis(trifluoromethyl)phenyl]ethanol Catalyzed by Engineered Ketoreductase

To a 30 mL reaction flask, 0.1 g of SEQ ID No: 56 wet cells was added, then 0.25 g of 3,5-bis(trifluoromethyl)acetophenone, 0.5 mL of isopropanol and 0.25 mL of NAD+(10 g/L of stock solution) were added to the reaction flask. The final reaction volume in the reaction flask was topped up to 5.0 mL with 0.1 M PB at pH 7, the final concentration of the substrate (3,5-bis(trifluoromethyl)acetophenone) in the reaction flask was 50 g/L, the final concentration of isopropanol was 10% v/v, and the final concentration of NAD+ was 0.5 g/L. The reaction flask was placed on an IKA magnetic stirrer at 30° C. with the stirring speed set to 400 rpm to start the reaction. After the reaction was carried out for 24 h, samples were taken from the reaction flask and processed and analyzed. The results are shown in Table 7.

TABLE 7

| | Wet cell | Conver- | ee of product (S)-1-[3,5- |
|---|---|---|---|
| Enzyme | loading | sion | bis(trifluoromethyl)phenyl]ethanol |
| SEQ ID No: 56 | 20 g/L | 99.3% | >99.9% |

### Analysis Method:

### Analysis Method for Conversion:

The chromatographic column was ZORBAX SB-C18 (150 m*4.6 mm*5 m), mobile phase: 60% acetonitrile+40% water, column temperature: 30° C., injection volume: 10 μL, flow rate: 1.0 ml/min, wavelength: 218 nm, where the retention time of (S)-1-[3,5-bis(trifluoromethyl)phenyl]ethanol was 5.0 min, the retention time of 3,5-bis(trifluoromethyl)acetophenone was 7.0 min.

### Chiral Analysis Method:

The chromatographic column was Agilent CYCLODEX-B 30 m*0.320 mm, 0.25 μm, the carrier gas was N2, the detector was FID, the inlet temperature was 200° C., the split ratio was 30:1, the detector temperature was 250° C., the injection volume was 1 μL, the column temperature was 115° C., the stopping time was 20 min, the retention time of (S)-1-[3,5-bis(trifluoromethyl) phenyl]ethanol was 8.3 min and the retention time of (R)-1-[3,5-bis(trifluoromethyl)phenyl]ethanol was 8.8 min.

### Example 24: Synthesis of (S)-1-tert-butoxycarbonyl-3-hydroxypiperidine Catalyzed by Engineered Ketoreductase

To a 30 mL reaction flask, 0.1 g of SEQ ID No: 102 bacterium was added, and then 0.25 g of N-Boc-3-piperidone, 0.5 mL of isopropanol and 0.25 mL of NAD+(10 g/L of stock solution) were added to the reaction flask. The final reaction volume in the reaction flask was topped up to 5.0 mL with 0.1 M PB at pH 7, the final concentration of the substrate (N-Boc-3-piperidone) in the reaction flask was 50 g/L, the final concentration of isopropanol was 10% v/v, and the final concentration of NAD+ was 0.5 g/L. The reaction flask was placed on an IKA magnetic stirrer at 30° C. with the stirring speed set to 400 rpm to start the reaction. After the reaction was carried out for 24 h, samples were taken from the reaction flask and processed and analyzed. The results are shown in Table 8.

TABLE 8

| | | | ee of product (S)- |
|---|---|---|---|
| | Wet cell | | 1-tert-butoxycarbonyl-3- |
| Enzyme | loading | Conversion | hydroxypiperidine |
| SEQ ID No: 102 | 20 g/L | 97.3% | 98.89% |

### Analysis Method:

The chromatographic column was Agilent DB-WAX 30 m*0.250 mm, 0.25 μm, the carrier gas was N2, the detector was FID, the inlet temperature was 250° C., the split ratio was 20:1, the detector temperature was 250° C., the injection volume was 3 μL, the column flow rate: 1.0 mL/min, the initial column temperature was 30° C., the temperature was ramped up to 150° C. at a rate of 20° C./min, kept for 5 min, then ramped up to 230° C. at a rate of 5° C./min and kept for 5 min, stopping time was 32 min, the retention time of N-tert-butoxycarbonyl-3-piperidone was 21.3 min for and the retention time of (S)-1-tert-butoxycarbonyl-3-hydroxypiperidine was 24.3 min.

### Example 25: Synthesis of (S)-(−)-3-chloro-1-phenyl-1-propanol Catalyzed by Engineered Ketoreductase

To a 30 mL reaction flask, 0.1 g of SEQ ID No: 34 wet cells was added, then 0.01 g of 3-chloropropiophenone, 0.5 mL of isopropanol and 0.25 mL (10 g/L of stock solution) of NAD+ were added to the reaction flask. The final reaction volume in the reaction flask was topped up to 5.0 mL with pure water, the final concentration of the substrate (3-chloropropiophenone) was 2 g/L, the final concentration of isopropanol was 10% v/v, and the final concentration of NAD+ was 0.5 g/L. The reaction flask was placed on an IKA magnetic stirrer at 30° C. with the stirring speed set to 400 rpm to start the reaction. After the reaction was carried out for 24 h, samples were taken from the reaction flask and processed and analyzed. The results are shown in Table 9.

TABLE 9

| | | | ee of product (S)- |
|---|---|---|---|
| | Wet cell | | (−)-3-chloro-1- |
| Enzyme | loading | Conversion | phenyl-1-propanol |
| SEQ ID No: 34 | 20 g/L | 66.17% | 88.44% |

### Analysis Method:

### Analysis Method for Conversion:

The chromatographic column was ZORBAX SB-C18 (150 m*4.6 mm*5 m), mobile phase: 50% acetonitrile+50% HClO₄, column temperature: 45° C., injection volume: 10 L, flow rate: 2.0 ml/min, wavelength: 215 nm, where the retention time of (S)-(−)-3-chloro-1-phenyl-1-propanol was 1.9 min, and the retention time of 3-chloro-1-phenyl-propanol was 2.7 min.

### Chiral Analysis Method:

Method of Sample pretreatment: 200 μL of sample was added with 50 μL of MSTFA and 30 μL of anhydrous pyridine in a 1.5 mL centrifuge tube and mixed well, and the reaction was shaken for 30 min. The column was an Agilent CP-Chirasil Dex CB (CP7502) 25 m*0.25 mm*0.25 μm, the carrier gas was N2, and the detector was FID. The inlet temperature was 250° C., the shunt ratio was 10:1, the detector temperature was 260° C., the column flow rate was 1.1 mL/min, the injection volume was 1 μL, the initial column temperature was 100° C., maintained for 20 min, ramped up to 120° C. at a rate of 5° C./min, maintained for 20 min, the stopping time was 44 min, the retention time of (S)-(−)-3-chloro-1-phenyl-1-propanol was 35.6 min and the retention time of (R)-(+)-3-chloro-1-phenyl-1-propanol was 38.8 min.

### Example 26: Synthesis of 2,7-dimethyl-3,6-octanediol Catalyzed by Engineered Ketoreductase

To a 30 mL reaction flask with a volume of, 0.125 g of SEQ ID No: 60 wet cells was added, then 0.05 g of 2,7-dimethyl-3,6-octanedione, 0.5 mL of isopropanol and 0.25 mL of NAD+(10 g/L of stock solution) was added to the reaction flask. The final reaction in the reaction flask was topped up to 5.0 mL with 0.1 M PB at pH 7, the final concentration of the substrate (2,7-dimethyl-3,6-octanedione) in the reaction flask was 10 g/L, the final concentration of isopropanol was 10% v/v, and the final concentration of NAD+ was 0.5 g/L. The reaction flask was placed on an IKA magnetic stirrer at 30° C. with the stirring speed set to 400 rpm to start the reaction. After the reaction was carried out for 18 h, samples were taken from the reaction flask and processed and analyzed. The results obtained are shown in Table 10. In addition, ketoreductase can catalyze 2,3-butanedione to produce (2S,3S)-2,3-butanediol; catalyze acetylacetone to produce (2S,4S)-2,4-pentanediol; catalyze 3,6-octanedione to produce (3S,6S)-3,6-octanediol; catalyze 3,5-heptanedione to (3S,5S)-3,5-heptanediol.

TABLE 10

| Enzyme | Wet cell loading | Conversion |
|---|---|---|
| SEQ ID No: 60 | 25 g/L | 97% |

### Analysis Method:

The chromatographic column was Agilent CP-Chirasil-Dex CB (CP7502) 25 m*0.25 mm*0.25 μm, the carrier gas was N2, the detector was FID, the inlet temperature was 250° C., the split ratio was 20:1, the detector temperature was 300° C., the injection volume was 1 μL, the column temperature was 40° C., kept for 0.5 min, followed heated to 77° C. at a rate of 10° C./min and kept for 0.5 min, then heated to 190° C. at a rate of 40° C./min and kept for 5.0 min, where the retention time of 2,7-dimethyl-3,6-octanedione was 8.6 min.

It should be understood that after reading the above contents of the present invention, those skilled in the art may make various modifications or changes to the present invention. And these equivalent forms also fall within the scope of the appended claims of the present invention.

1. An engineered ketoreductase polypeptide that catalyzes the reduction of ethyl 4-chloroacetoacetate to produce ethyl (R)-4-chloro-3-hydroxybutyrate with an ee value of at least 99%, the polypeptide comprising an amino acid sequence having at least 90% sequence identity to the reference sequence SEQ ID NO: 2 and at least two residue differences at residue positions X204, X211 compared to SEQ ID NO: 2, wherein the amino acid at residue position X204 is I and the amino acid at residue position X211 is F.

2. An engineered ketoreductase polypeptide, which is capable of converting ethyl 4-chloroacetoacetate to ethyl (R)-4-chloro-3-hydroxybutyrate with better catalytic activity and/or stability than SEQ ID NO: 2, wherein the amino acid sequence is selected from the group consisting of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128.

3. The ketoreductase polypeptide as claimed in claim 1, wherein reaction conditions include 5 g/L-426 g/L of ethyl 4-chloroacetoacetate loading, pH of 5.0-8.0, and temperature of 10-80° C.

4. A polypeptide immobilized on a solid material by chemical bonding or physical adsorption, wherein the polypeptide is selected from the ketoreductase polypeptides of claim 1.

5. A polynucleotide encoding the polypeptide of claim 1.

6. The polynucleotide as claimed in claim 5, wherein the polynucleotide sequence is selected from the group consisting of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127.

7. An expression vector, the vector comprises the polynucleotide of claim 5.

8. The expression vector of claim 6, which comprises a plasmid, a cosmid, a bacteriophage or a viral vector.

9. A host cell, which comprising the expression vector of claim 7, wherein the host cell is preferably E. coli.

10. A method of preparing a ketoreductase polypeptide, which comprises the steps of culturing the host cell of claim 9 and obtaining the ketoreductase polypeptide from the culture.

11. A ketoreductase catalyst obtainable from the method of claim 10, wherein the ketoreductase catalyst comprises cells or culture fluid containing the ketoreductase polypeptide obtained from the culture, or an article processed therewith, wherein the article refers to an extract obtained from the host cell, an isolated product obtained by isolating or purifying a ketoreductase from the extract, or an immobilized product obtained by immobilizing the host cell, an extract thereof, or isolated product of the extract.

12. A process for the preparation of compounds of structural formula (I): 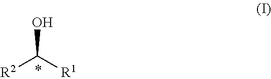
The alcohol product of said structural formula (I) has the indicated stereochemical configuration shown at the chiral center marked with an *; the alcohol product of said structure (I) is in enantiomeric excess over the other isomer; where
R¹ is optionally substituted or unsubstituted aryl or heteroaryl, or optionally substituted or unsubstituted C₁-C₈ hydrocarbon group, or may be a cyclic hydrocarbon group or a heterocyclic group;
R² is an optionally substituted or unsubstituted C₁-C₆ hydrocarbon group, a halogen (such as —F, —Cl, —Br and —I), an alkenyl, an alkynyl, an aryl, a heteroaryl, —NO₂, —NO, —SO₂R′ or —SOR′, —SR′, —NR′R′, —OR′, —CO₂R′ or —COR′, —C(O)NR′, —SO₂NH₂ or —SONH₂, —CN, —CF₃; wherein each R′ is independently selected from —H, C₁-C₄ hydrocarbon group, halogen, C₁-C₈ hydrocarbon group, C₂-C₁₂ alkenyl group, C₂-C₁₂ alkynyl group, cyclic hydrocarbon group, aryl group or heterocyclic group;
wherein the process comprises contacting a carbonyl substrate of structural formula (II) 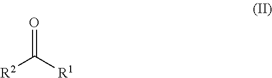
with the engineered polypeptide of claim 1.

13. The process as claimed in claim 12, wherein the products of structural formula (I) are 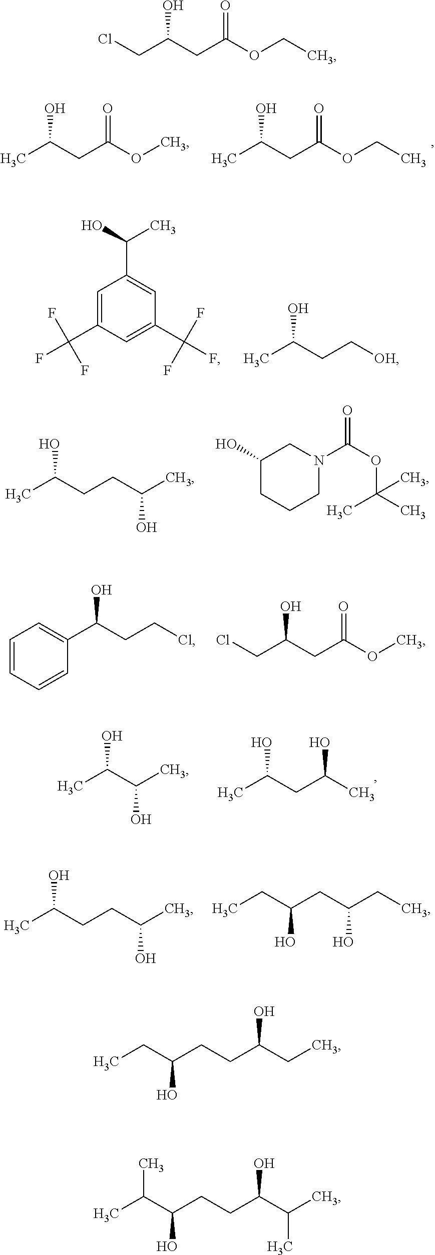
and the carbonyl substrates of structural formula (II) are

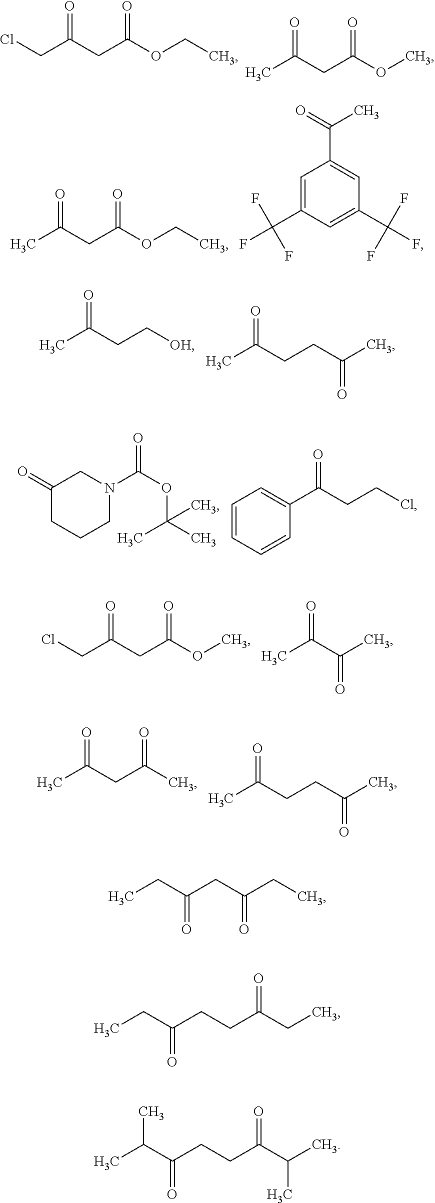

14. A process for the preparation of the compound ethyl (R)-(+)-4-chloro-3-hydroxybutyrate: 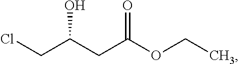
wherein the process comprises contacting the carbonyl substrate ethyl 4-chloroacetoacetate: 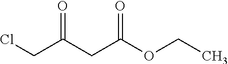
with the engineered ketoreductase polypeptide of claim 1.

15. The process as claimed in claim 12, wherein the reaction solvent comprises water, toluene, isopropanol, acetone; temperature is 10° C. to 80° C. and pH is 5.0 to 8.0.


SEQUENCE LISTING (128 sequences)

SEQ ID NO: 1 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatctggatgacgatcgtctggcactggcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttttcatgatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 2 - protein, 346 aa, Rhodococcus erythropolis
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDLDDDRLALAREVGADAAVKSGAGAADAIRELTGGEGATAVFDFVGAQSTIDTAQQVVAIDGHISVVGIHAGAHAKVGFFMIPFGASVVTPYWGTRSELMDVVDLARAGRLDIHTETFTLDEGPTAYRRLREGSIRGRGVVVPG

SEQ ID NO: 3 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacctttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 4 - protein, 346 aa, synthetic construct
MKALQYTEIGSEPVVVDVPTPAPGPGEILLKVTAAGLCHSDLFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 5 - DNA, 1038 bp, synthetic construct
  source 1..1038: mol_type = other DNA
atgaaagcactgcaatataccgaaattggctccgaaccggtggtcgttgatatcccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 6 - protein, 346 aa, synthetic construct
MKALQYTEIGSEPVVVDIPTPAPGPGEILLKVTAAGLCHSDIFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 7 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcaagaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 8 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFKTGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 9 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatcagtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 10 - protein, 346 aa, synthetic construct
MKALQYTEIGSEPVVVDVPTPAPGPGEILLKVTAAGLCHSDIFVMDMPAEQYQYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 11 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatgggtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 12 - protein, 346 aa, synthetic construct
MKALQYTEIGSEPVVVDVPTPAPGPGEILLKVTAAGLCHSDIFVMDMPAEQYGYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 13 - DNA, 1038 bp, synthetic construct
  source 1..1038: mol_type = other DNA
atgaaagcactgcaatataccgaaattggctccgaaccggtggtcgttgatgttccgacgccgaagccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 14 - protein, 346 aa, synthetic construct
MKALQYTEIGSEPVVVDVPTPKPGPGEILLKVTAAGLCHSDIFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 15 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatccttacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 16 - protein, 346 aa, synthetic construct
MKALQYTEIGSEPVVVDVPTPAPGPGEILLKVTAAGLCHSDIFVMDMPAEQYPYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 17 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatacgtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 18 - protein, 346 aa, synthetic construct
MKALQYTEIGSEPVVVDVPTPAPGPGEILLKVTAAGLCHSDIFVMDMPAEQYTYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 19 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtattcttacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 20 - protein, 346 aa, synthetic construct
MKALQYTEIGSEPVVVDVPTPAPGPGEILLKVTAAGLCHSDIFVMDMPAEQYSYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 21 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtcgtggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 22 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGRGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 23 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtaagggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 24 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGKGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 25 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgctggcctatcgtcgcctgcgtgaaggtagcatccgtggtcgtggtgtcgttgtgccgggc

SEQ ID NO: 26 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPLAYRRLREGSIRGRGVVVPG

SEQ ID NO: 27 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccggaggcctatcgtcgcctgcgtgaaggtagcatccgtggtcgtggtgtcgttgtgccgggc

SEQ ID NO: 28 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPEAYRRLREGSIRGRGVVVPG

SEQ ID NO: 29 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctcacctgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 30 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAHLGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 31 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattcgtccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 32 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGIRPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 33 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtatttgtccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 34 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGICPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 35 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattagtccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 36 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGISPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 37 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattcatccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 38 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGIHPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 39 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttggcgatatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 40 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFGDIPFGASVVTPYWGTRSELMDVVDLARAGRLDIHTETFTLDEGPTAYRRLREGSIRGRGVVVPG

SEQ ID NO: 41 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaagtgggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgcggcgattcgtgaactgacgggcggtgaaggcgcaaccgctgtgtttgacttcgttggtgcgcaaagcacgatcgataccgcccagcaagtcgtggcagtggacggccatatctctgttgtcggcattcatgcgggtgcccacgcaaaagtgggctttcagaacatcccgtttggtgcgtcagtggttacgccgtattggggtacccgctcggaactgatggaagtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 42 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFEVGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAAAAIRELTGGEGATAVFDFVGAQSTIDTAQQVVAVDGHISVVGIHAGAHAKVGFQNIPFGASVVTPYWGTRSELMEVVDLARAGRLDIHTETFTLDEGPTAYRRLREGSIRGRGVVVPG

SEQ ID NO: 43 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaagtgggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagcgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgcggcgattcgtgaactgacgggcggtgaaggcgcaaccgctgtgtttgacttcgttggtgcgcaaagcacgatcgataccgcccagcaagtcgtggcagtggacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 44 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFEVGDAVAVYGPWGCGACHACARGRENYCTRAAELGITPPGLGSPGSMAEYMIVDSARHLVPIGDLDPVAAAPLTDAGLTPYHAISRVLPLLGPGSTAVVIGVGGLGHVGIQILRAVSAARVIAVDIDDDRLAFAREVGADAAVKSGAGAAAAIRELTGGEGATAVFDFVGAQSTIDTAQQVVAVDGHISVVGIHAGAHAKVGFQNIPFGASVVTPYWGTRSELMDVVDLARAGRLDIHTETFTLDEGPTAYRRLREGSIRGRGVVVPG

SEQ ID NO: 45 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaagtgggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcagtggacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 46 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFEVGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAVDGHISVVGIHAGAHAKVGFQNIPFGASVVTPYWGTRSELMDVVDLARAGRLDIHTETFTLDEGPTAYRRLREGSIRGRGVVVPG

SEQ ID NO: 47 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaagtgggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagcgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgcggcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 48 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFEVGDAVAVYGPWGCGACHACARGRENYCTRAAELGITPPGLGSPGSMAEYMIVDSARHLVPIGDLDPVAAAPLTDAGLTPYHAISRVLPLLGPGSTAVVIGVGGLGHVGIQILRAVSAARVIAVDIDDDRLAFAREVGADAAVKSGAGAAAAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 49 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaagtgggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgcggcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 50 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFEVGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAAAAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 51 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaagtgggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 52 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFEVGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 53 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaagtgggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagcgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 54 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFEVGDAVAVYGPWGCGACHACARGRENYCTRAAELGITPPGLGSPGSMAEYMIVDSARHLVPIGDLDPVAAAPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 55 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaagtgggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgcggcgattcgtgaactgacgggcggtgaaggcgcaaccgctgtgtttgacttcgttggtgcgcaaagcacgatcgataccgcccagcaagtcgtggcagtggacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 56 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFEVGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAAAAIRELTGGEGATAVFDFVGAQSTIDTAQQVVAVDGHISVVGIHAGAHAKVGFQNIPFGASVVTPYWGTRSELMDVVDLARAGRLDIHTETFTLDEGPTAYRRLREGSIRGRGVVVPG

SEQ ID NO: 57 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaagtgggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagcgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgcggcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggaagtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 58 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFEVGDAVAVYGPWGCGACHACARGRENYCTRAAELGITPPGLGSPGSMAEYMIVDSARHLVPIGDLDPVAAAPLTDAGLTPYHAISRVLPLLGPGSTAVVIGVGGLGHVGIQILRAVSAARVIAVDIDDDRLAFAREVGADAAVKSGAGAAAAIRELTGGEGATAVFDFVGAQSTIDTAQQVVAIDGHISVVGIHAGAHAKVGFQNIPFGASVVTPYWGTRSELMEVVDLARAGRLDIHTETFTLDEGPTAYRRLREGSIRGRGVVVPG

SEQ ID NO: 59 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaagtgggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggaagtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 60 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFEVGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMEVVDLARAGRLDIHTETFTLDEGPTAYRRLREGSIRGRGVVVPG

SEQ ID NO: 61 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaagtgggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcagtggacggccatatctctgttgtcggcattcatgcgggtgcccacgcaaaagtgggctttcagaacatcccgtttggtgcgtcagtggttacgccgtattggggtacccgctcggaactgatggaagtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 62 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFEVGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAVDGHISVVGIHAGAHAKVGFQNIPFGASVVTPYWGTRSELMEVVDLARAGRLDIHTETFTLDEGPTAYRRLREGSIRGRGVVVPG

SEQ ID NO: 63 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatccgtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatgccggtcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 64 - protein, 346 aa, synthetic construct
MKALQYTEIGSEPVVVDVPTPAPGPGEILLKVTAAGLCHSDIFVMDMPAEQYPYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNMPVGASVVTPYWGTRSELMDVVDLARAGRLDIHTETFTLDEGPTAYRRLREGSIRGRGVVVPG

SEQ ID NO: 65 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatccgtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacataccgcacggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 66 - protein, 346 aa, synthetic construct
MKALQYTEIGSEPVVVDVPTPAPGPGEILLKVTAAGLCHSDIFVMDMPAEQYPYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPHGASVVTPYWGTRSELMDVVDLARAGRLDIHTETFTLDEGPTAYRRLREGSIRGRGVVVPG

SEQ ID NO: 67 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatcattacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacataccggtcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 68 - protein, 346 aa, synthetic construct
MKALQYTEIGSEPVVVDVPTPAPGPGEILLKVTAAGLCHSDIFVMDMPAEQYHYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPVGASVVTPYWGTRSELMDVVDLARAGRLDIHTETFTLDEGPTAYRRLREGSIRGRGVVVPG

SEQ ID NO: 69 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatccgtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacataccggtcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 70 - protein, 346 aa, synthetic construct
MKALQYTEIGSEPVVVDVPTPAPGPGEILLKVTAAGLCHSDIFVMDMPAEQYPYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPVGASVVTPYWGTRSELMDVVDLARAGRLDIHTETFTLDEGPTAYRRLREGSIRGRGVVVPG

SEQ ID NO: 71 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatccgtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacataccgcgcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 72 - protein, 346 aa, synthetic construct
MKALQYTEIGSEPVVVDVPTPAPGPGEILLKVTAAGLCHSDIFVMDMPAEQYPYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPRGASVVTPYWGTRSELMDVVDLARAGRLDIHTETFTLDEGPTAYRRLREGSIRGRGVVVPG

SEQ ID NO: 73 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtataactacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatgccggtcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 74 - protein, 346 aa, synthetic construct
MKALQYTEIGSEPVVVDVPTPAPGPGEILLKVTAAGLCHSDIFVMDMPAEQYNYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNMPVGASVVTPYWGTRSELMDVVDLARAGRLDIHTETFTLDEGPTAYRRLREGSIRGRGVVVPG

SEQ ID NO: 75 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatcattacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacataccgcgcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 76 - protein, 346 aa, synthetic construct
MKALQYTEIGSEPVVVDVPTPAPGPGEILLKVTAAGLCHSDIFVMDMPAEQYHYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPRGASVVTPYWGTRSELMDVVDLARAGRLDIHTETFTLDEGPTAYRRLREGSIRGRGVVVPG

SEQ ID NO: 77 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatacctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacctgccggtcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 78 - protein, 346 aa, synthetic construct
MKALQYTEIGSEPVVVDVPTPAPGPGEILLKVTAAGLCHSDIFVMDMPAEQYTYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNLPVGASVVTPYWGTRSELMDVVDLARAGRLDIHTETFTLDEGPTAYRRLREGSIRGRGVVVPG

SEQ ID NO: 79 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtataactacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacataccgcacggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 80 - protein, 346 aa, synthetic construct
MKALQYTEIGSEPVVVDVPTPAPGPGEILLKVTAAGLCHSDIFVMDMPAEQYNYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPHGASVVTPYWGTRSELMDVVDLARAGRLDIHTETFTLDEGPTAYRRLREGSIRGRGVVVPG

SEQ ID NO: 81 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatccgtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacctgccggtcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 82 - protein, 346 aa, synthetic construct
MKALQYTEIGSEPVVVDVPTPAPGPGEILLKVTAAGLCHSDIFVMDMPAEQYPYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNLPVGASVVTPYWGTRSELMDVVDLARAGRLDIHTETFTLDEGPTAYRRLREGSIRGRGVVVPG

SEQ ID NO: 83 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatccgtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacgtaccgcgcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 84 - protein, 346 aa, synthetic construct
MKALQYTEIGSEPVVVDVPTPAPGPGEILLKVTAAGLCHSDIFVMDMPAEQYPYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNVPRGASVVTPYWGTRSELMDVVDLARAGRLDIHTETFTLDEGPTAYRRLREGSIRGRGVVVPG

SEQ ID NO: 85 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatacctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacgtaccgcgcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 86 - protein, 346 aa, synthetic construct
MKALQYTEIGSEPVVVDVPTPAPGPGEILLKVTAAGLCHSDIFVMDMPAEQYTYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNVPRGASVVTPYWGTRSELMDVVDLARAGRLDIHTETFTLDEGPTAYRRLREGSIRGRGVVVPG

SEQ ID NO: 87 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatcgctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacataccgcgcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 88 - protein, 346 aa, synthetic construct
MKALQYTEIGSEPVVVDVPTPAPGPGEILLKVTAAGLCHSDIFVMDMPAEQYRYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPRGASVVTPYWGTRSELMDVVDLARAGRLDIHTETFTLDEGPTAYRRLREGSIRGRGVVVPG

SEQ ID NO: 89 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatccgtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatgccgcgcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 90 - protein, 346 aa, synthetic construct
MKALQYTEIGSEPVVVDVPTPAPGPGEILLKVTAAGLCHSDIFVMDMPAEQYPYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNMPRGASVVTPYWGTRSELMDVVDLARAGRLDIHTETFTLDEGPTAYRRLREGSIRGRGVVVPG

SEQ ID NO: 91 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatacctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacataccggtcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 92 - protein, 346 aa, synthetic construct
MKALQYTEIGSEPVVVDVPTPAPGPGEILLKVTAAGLCHSDIFVMDMPAEQYTYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPVGASVVTPYWGTRSELMDVVDLARAGRLDIHTETFTLDEGPTAYRRLREGSIRGRGVVVPG

SEQ ID NO: 93 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatccgtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacgtaccgcacggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 94 - protein, 346 aa, synthetic construct
MKALQYTEIGSEPVVVDVPTPAPGPGEILLKVTAAGLCHSDIFVMDMPAEQYPYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNVPHGASVVTPYWGTRSELMDVVDLARAGRLDIHTETFTLDEGPTAYRRLREGSIRGRGVVVPG

SEQ ID NO: 95 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtataagtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacataccggtcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 96 - protein, 346 aa, synthetic construct
MKALQYTEIGSEPVVVDVPTPAPGPGEILLKVTAAGLCHSDIFVMDMPAEQYKYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPVGASVVTPYWGTRSELMDVVDLARAGRLDIHTETFTLDEGPTAYRRLREGSIRGRGVVVPG

SEQ ID NO: 97 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatccgtacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacgtaccggtcggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 98 - protein, 346 aa, synthetic construct
MKALQYTEIGSEPVVVDVPTPAPGPGEILLKVTAAGLCHSDIFVMDMPAEQYPYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNVPVGASVVTPYWGTRSELMDVVDLARAGRLDIHTETFTLDEGPTAYRRLREGSIRGRGVVVPG

SEQ ID NO: 99 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatgggtatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 100 - protein, 346 aa, synthetic construct
MKALQYTEIGSEPVVVDVPTPAPGPGEILLKVTAAGLCHSDIFVMGMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 101 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatgtttatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 102 - protein, 346 aa, synthetic construct
MKALQYTEIGSEPVVVDVPTPAPGPGEILLKVTAAGLCHSDIFVMFMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 103 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggttatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 104 - protein, 346 aa, synthetic construct
MKALQYTEIGSEPVVVDVPTPAPGPGEILLKVTAAGLCHSDIFVMVMPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 105 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgcgtgccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 106 - protein, 346 aa, synthetic construct
MKALQYTEIGSEPVVVDVPTPAPGPGEILLKVTAAGLCHSDIFVMDMRAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 107 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatattccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 108 - protein, 346 aa, synthetic construct
MKALQYTEIGSEPVVVDVPTPAPGPGEILLKVTAAGLCHSDIFVMDIPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 109 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatagtccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 110 - protein, 346 aa, synthetic construct
MKALQYTEIGSEPVVVDVPTPAPGPGEILLKVTAAGLCHSDIFVMDSPAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 111 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgcatgccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 112 - protein, 346 aa, synthetic construct
MKALQYTEIGSEPVVVDVPTPAPGPGEILLKVTAAGLCHSDIFVMDMHAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 113 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgcatgccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaagcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 114 - protein, 346 aa, synthetic construct
MKALQYTEIGSEPVVVDVPTPAPGPGEILLKVTAAGLCHSDIFVMDMHAEQYIYGLPLTLGHEGVGTVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGESATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 115 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtgttgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 116 - protein, 346 aa, synthetic construct
MKALQYTEIGSEPVVVDVPTPAPGPGEILLKVTAAGLCHSDIFVMDMPAEQYIYGLPLTLGHEGVGVVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 117 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtattgttgccgaactgggcgcaggtgtgacgggcttcgaaaccggtgatgcagttgcagtctatggtccgtggggctgcggtgcatgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 118 - protein, 346 aa, synthetic construct
MKALQYTEIGSEPVVVDVPTPAPGPGEILLKVTAAGLCHSDIFVMDMPAEQYIYGLPLTLGHEGVGIVAELGAGVTGFETGDAVAVYGPWGCGACHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 119 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtgcatgtcgtgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 120 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGACRACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 121 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtcgttgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 122 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGRCHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 123 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggttattgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 124 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGYCHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 125 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggttgttgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 126 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGCCHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG

SEQ ID NO: 127 - DNA, 1038 bp, synthetic construct
  source 1..1038: mol_type = other DNA
atgaaagcactgcaatataccgaaattggctccgaaccggtggtcgttgatgttccgacgccggctccgggtccgggtgaaatcctgctgaaagtgaccgcggccggcctgtgtcatagcgacatttttgttatggatatgccggccgaacagtatatctacggtctgccgctgacgctgggtcacgaaggcgtgggtaccgttgccgaactgggcgcaggtgtgacgggcttcgaaaccggtgatgcagttgcagtctatggtccgtggggctgcggtcattgtcatgcatgcgctcgtggtcgcgaaaactactgcacgcgtgcagctgaactgggtattaccccgccgggtctgggtagcccgggttctatggcggaatatatgattgtggattccgcacgtcatctggttccgatcggcgacctggatccggtcgcagcagtgccgctgacggatgctggtctgaccccgtaccacgcaattagtcgtgtgctgccgctgctgggtccgggttccaccgcagtggttatcggtgtcggcggtctgggtcacgtgggtattcagatcctgcgtgcagttagtgcagctcgcgttatcgcggtcgatatcgatgacgatcgtctggcattcgcacgcgaagttggtgcagatgcggccgtcaaatcaggcgctggtgcagctgacgcgattcgtgaactgacgggcggtgaaggcgcaaccgctgtgtttgacttcgttggtgcgcaaagcacgatcgataccgcccagcaagtcgtggcaattgacggccatatctctgttgtcggcattcatgcgggtgcccacgcaaaagtgggctttcagaacatcccgtttggtgcgtcagtggttacgccgtattggggtacccgctcggaactgatggacgtcgtggatctggctcgtgcaggccgcctggatattcacaccgaaacgttcaccctggacgaaggtccgaccgcctatcgtcgcctgcgtgaaggtagcatccgtggtcgtggtgtcgttgtgccgggc

SEQ ID NO: 128 - protein, 346 aa, synthetic construct
MKALQYTEIGSEPVVVDVPTPAPGPGEILLKVTAAGLCHSDIFVMDMPAEQYIYGLPLTLGHEGVGTVAELGAGVTGFETGDAVAVYGPWGCGHCHACARGRENYCTRAAELGITPPGLGSPGSMAEYMIVDSARHLVPIGDLDPVAAVPLTDAGLTPYHAISRVLPLLGPGSTAVVIGVGGLGHVGIQILRAVSAARVIAVDIDDDRLAFAREVGADAAVKSGAGAADAIRELTGGEGATAVFDFVGAQSTIDTAQQVVAIDGHISVVGIHAGAHAKVGFQNIPFGASVVTPYWGTRSELMDVVDLARAGRLDIHTETFTLDEGPTAYRRLREGSIRGRGVVVPG